(19) United States
(12) Patent Application Publication
(10) Pub. No.: US 2020/0184419 A1
(43) Pub. Date: Jun. 11, 2020

(54) TRACEABILITY OF SWINE TISSUE

(71) Applicant: Australian Pork Limited, Barton, Australian Capital Territory (AU)

(72) Inventors: John Roger WATLING, Jandakot, Western Australia (AU); Garry LEE, Gwelup, Western Australia (AU)

(21) Appl. No.: 16/608,958

(22) PCT Filed: Apr. 30, 2018
(86) PCT No.: PCT/AU2018/050396

§ 371 Date: Oct. 28, 2019

(30) Foreign Application Priority Data
Apr. 28, 2017 (AU) 2017100469

(51) Int. Cl. G06Q 10/10; G06Q 10/08; G06Q 50/02; G06F 16/953; G01N 33/12; G01N 21/73

(52) U.S. Cl. CPC G06Q 10/10; G06Q 10/087; G06Q 50/02; G06Q 30/0185; G01N 33/12; G01N 21/73; G06F 16/953

(57) ABSTRACT

The present invention relates to methods and systems for reporting the identity of a sample sourced from an animal. In particular, the present invention relates to reporting the identity of a sample taken from a pig animal of the species Sus scrofa. More particularly, the invention relates to methods and systems for reporting the identity of an unknown pig animal sample comprising registering reference samples in a database through a register; recording data representing the reference samples; recording data representing the unknown sample and comparing the data to assess the identity of the unknown pig sample through the register.

### TECHNICAL FIELD

The present invention relates to methods and systems for reporting the identity of a sample sourced from an animal. In particular, the present invention relates to assessing a sample taken from a pig animal of the species Sus scrofa.

### BACKGROUND

With increasing demand for high quality product and the globalisation of food supply chains, the ability to be able to rapidly detect and mitigate product integrity risks in the food industry is essential. As a result, research and development programs are being conducted to develop and implement traceability systems. PigPass is a national tracking system used for movement reporting of all pigs in Australia and is supported by the completion of national vendor declarations. PigPass national vendor declarations (NVDs) link consignments of pigs back to their last property of origin using the property identification code (PIC) with this information then linked to the processor's system when pigs arrive at the processing establishment and scheduled for slaughter. These linkages can be used to enable traceability to property of origin for a carcase and may extend to boxed product and retail packs for fresh pork. For this system to enable traceback to region and/or property of origin for unpackaged or bulk packaged pig tissues, further technological developments are required.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY

Described herein is an electronic chemical traceability system, known as Physi-Trace™, that has been developed by the Australian pork industry through Australian Pork Limited (APL).

Features of the Pork Supply Chain Integrity Program including the Physi-Trace™ system are depicted in FIG. 1. The system can be used to validate the origin and/or verify a label claim of a pork product and may be supported by its integrated system management procedures used to manage the existing industry quality assurance program, the Australian Pork Industry Quality Assurance system (APIQ✓®) and PigPass. The Physi-Trace™ traceability system is underpinned by scientific technologies associated with the analysis of the elemental composition of samples.

Validation works on the basis of comparing elemental distribution patterns of an unknown sample with a database of reference patterns. This involves the collection of reference samples with known identity e.g. geographic region of origin and/or property of origin. Unique multi-element chemical profiles of pigs from different farms are incorporated into a database that can be used to assess the identity/provenance, e.g. geographic region of origin and/or property of origin, of an unknown pig sample by comparison against the known identity of the reference samples. As chemical signatures are difficult to falsify, chemical traceability is an unambiguous and robust means of assessing the identity of an unknown sample.

The Australian pork industry currently exports about 10% of its production to export markets including Singapore, New Zealand and Hong Kong. It is therefore imperative that the Australian pork industry has systems in place to rapidly trace product back to source in the unlikely event of a food safety, residue or animal disease issue to minimise risks associated with loss of market access.

In the event of a food safety incident or any other incident where traceability is required, the Physi-Trace™ system can be used to identify sources of alleged “suspect” product. This identification enables un-affected product, producers and processors to be excluded from further investigations facilitating market re-entry. In a simulated traceback exercise, a successful traceback was completed within 24 hours from an operational perspective. The National Livestock Traceability Performance Standards require that within 24 hours of the relevant state or territory Chief Veterinary Officer (or their delegate) in the jurisdiction where the specified animal(s) is located or been traced to being notified, it must be possible to identify the location(s) where specified animal(s) had resided during the previous 30 days and location(s) of all susceptible animals that resided concurrently and/or subsequently on any of the properties that the specified animal(s) had resided within the last 30 days.

As described herein, the Physi-Trace™ system has been used to trace the identity (provenance) of, for example, pork muscle, offal and processed products. In the case of pork muscle it is possible to trace the fresh pork back to not only the processor of origin, but to the property of origin (where the property of origin is linked to a tattoo code, PIC and registered with PigPass). In the case of offal, it has been demonstrated that it is possible to trace tongue, stomach, heart, liver and kidney tissues to a broad geographic region of origin. By applying offal-specific conversion ratios to normalise the multi-elemental profile of these different offal tissue types with their muscle-equivalent concentrations, integration of the different offal tissue types into a single muscle specific database has been possible. This advantageously provides means of tracing samples to a region of origin and/or/property of origin using only one database. In the case of processed meat products, such as whole muscle ham and bacon products, the methods and systems described herein have been used to provide an assessment of a country of origin label claim (e.g. PorkMark or Product of Australia).

The addressee would recognise that the present disclosure contemplates methods and systems of commercial importance. The methods and systems of the Physi-Trace™ validation tool as described herein provide a number of benefits. The tool can be implemented into any establishment at low cost without changing existing systems or work practices and does not require expensive capital equipment to be put in place by a processor. The tool is industry driven and administered. It uses internationally recognised traceability technology to provide traceability reporting and a fully robust traceability system to underpin Australian pork product integrity in all markets.

As would be recognised by the addressee, methods and systems for reporting the identity of unknown pig samples involves a number of participants (herein after “users”). Users of the methods and systems of the Physi-Trace™ database and validation tool described herein involve a number of participants. These users may be:

Supply chain users such as producers, processors and authorised third parties.

Government users such as the Australian Department of Agriculture and Water Resources, Biosecurity Animal Division, Exports Division and National Residue Survey and State and Territory Departments.

Other users may include:

Contractors (e.g laboratories and scientists who provide analytical services).

State and Territory organisations,

Veterinarians;

Commercial companies and

Industry associations and bodies.

The Physi-Trace™ system as a whole is administered by Australian Pork Limited. It will be appreciated that the Physi-Trace™ system is governed by business rules that have been endorsed by representatives from the seven Australian pork export establishments. Each user is appropriately authorised to upload data into the Physi-Trace™ database and validation tool according to these business rules.

The present disclosure relates to a method of reporting the identity of an unknown pig sample, the method comprising:
a) registering a plurality of samples referenced to an individual pig animal or a group of pig animals in a database;
b) recording data representing the plurality of reference samples against the register;
c) recording data representing an unknown pig sample in the database;
d) comparing data representing the unknown pig sample with the data representing the plurality of reference samples to thereby assess the identity of the unknown pig sample through the register; and
e) generating a report providing an assessment of the identity of the unknown pig sample.

The present disclosure also relates to a system for reporting the identity of an unknown pig sample, the system comprising:
a) means for registering a plurality of samples referenced to an individual pig animal or a group of pig animals;
b) means for recording data representing the plurality of reference samples against the register;
c) means for recording data representing the unknown pig sample;
d) means for comparing data representing the unknown pig sample with the data representing the plurality of reference samples to thereby identify the unknown pig sample; and
e) means for generating a report providing an assessment of the unknown pig sample.

The present disclosure also relates to a computer implemented system for reporting the identity of an unknown pig sample, the computer implemented system comprising a processor configured to:
a) register a plurality of samples referenced to an individual pig animal or a group of pig animals in a database;
b) record data representing the plurality of reference samples against the register;
c) record data representing the unknown pig sample;
d) compare data representing the unknown pig sample with the data representing the plurality of reference samples to thereby assess the identify the unknown pig sample; and/or
e) generate a report providing an assessment of the unknown pig sample.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein “Australian Pork Limited (APL) PigPass Registration Number” or “APL PigPass Registration Number” is an internal reference number granted to properties registered in PigPass by Australian Pork Limited to link a sample with a property of origin. It may be used separately in the Physi-Trace™ system. Or, it may be used in conjunction with a tattoo/tattoo code or property identification code (PIC) where it may be used to differentiate properties which have the same PIC or tattoo(s).

Throughout the present specification, various aspects and components of the disclosure can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 2, from 1 to 3, from 1 to 4, from 2 to 3, from 2 to 4, from 2 to 5, from 3 to 4 etc., as well as individual and partial numbers within the recited range, for example, 1, 2, 3, 4, and 5. This applies regardless of the breadth of the disclosed range. Where specific values are required, these will be indicated in the specification.

### BRIEF DESCRIPTION OF DRAWINGS

FIG. 1: Diagram showing features of the Pork Supply Chain Integrity Program.

FIG. 2: A flow diagram of one example of a method of reporting the identity of an unknown pig sample.

FIG. 3A: Schematic diagram of one example of a system for reporting the identity of an unknown pig sample.

FIG. 3B: Schematic diagram of another example of a system for reporting the identity of an unknown pig sample which is a variation of the example shown in FIG. 3A.

FIGS. 4A and 4B: A flow diagram showing an algorithm to reporting the identity of an unknown pig sample.

FIG. 5: Schematic diagram of one example of a system for reporting the identity of an unknown pig sample.

FIG. 6: Schematic diagram of another example of a system for reporting the identity of an unknown pig sample.

FIG. 7: Discriminant plot detailing classification of unknown sample to tattoo code Farm W.

FIG. 8: Discriminant plot detailing classification of unknown sample to tattoo code Farm X.

FIG. 9: Region of origin assessment of swine muscle tissue from North-Eastern Australia (A), South-Eastern Australia (B) and Western Australia (C). Leave one out cross validation of 100% achieved. Model developed using Rb, Se, As, W, Co, Mg, V, Hf, K, Pd, Mn, Nb, Ce, Ti, Zn, Zr, Er, Sb and U in order of importance.

FIG. 10: State of origin assessment of swine kidney from Western Australia (A), Queensland (B), New South Wales, South Australia and Victoria (C). Leave one out cross validation of 88.98% achieved. Model developed using Rb, Sr, Cd, Pb, Co, Si, Li, Yb, Cr, Ge, Cs, Sb, Ba, Ca, Na, Lu, In, Dy, Hf and As in order of importance.

FIG. 11: South Eastern Australian state of origin for swine kidney from New South Wales (A), Victoria (B) and South Australia (C). Leave one out cross validation of 90.41% achieved. Model developed using Dy, Lu, Rb, Cd, Sb, U, S, P, Li, Si, Yb, Zn, La and Ca in order of importance.

FIG. 12: State of origin assignment for New South Wales (NSW) and South Australian (SA) swine heart samples.

FIG. 13: Farm of origin assessment of Western Australian swine tongue samples. Leave one out cross validation of 96.4% achieved. Model developed using Cs, B, Sr, Ti, Lu, La, As, Tl, Se, Sm and Li in order of importance.

FIG. 14: Farm of origin assessment for Queensland swine stomach samples. Leave one out cross validation of 96.2% achieved. Model developed using Cs, Co, Sr, Rb, Se, K, B, Bi, Er, S, Fe and Ge in order of importance.

FIG. 15: Farm of origin assessment of South Eastern Australian swine. Leave one out cross validation of 83.56% achieved. Model developed using Cs, Rb, Tl, Zr, V, Hg, Se, Ag, Co, Bi, K, P, Hf, Cd, Tm, Ni, Ce, Pd, Fe and Mo in order of importance.

FIG. 16: Agglomerative hierarchical clustering diagram of multi-element signatures for immunocastrated male and female swine tongue tissue.

FIG. 17: Agglomerative hierarchical clustering diagram of multi-element signatures for immunocastrated male and female swine stomach tissue.

FIG. 18: Agglomerative hierarchical clustering diagram of multi-element signatures for immunocastrated male and female swine muscle tissue.

FIG. 19: Agglomerative hierarchical clustering diagram of multi-element signatures for immunocastrated male and female swine kidney tissue.

FIG. 20: Intra versus Inter-farm variation in the multi-elemental profile of swine muscle. F1C—farm one immunocastrated males F1F—farm once females, F2M—farm one males, F2F—farm two female.

FIG. 21: Intra versus Inter-farm variation in the multi-elemental profile of swine stomach. F1C—farm one immunocastrated males, F1F—farm once females, F2M—farm one males, F2F—farm two female.

FIG. 22: Intra versus Inter-farm variation in the multi-elemental profile of swine kidney. F1C—farm one immunocastrated males, F1F—farm once females, F2M—farm one males, F2F—farm two female.

FIG. 23: Tissue of origin classification for swine muscle, tongue, stomach, heart liver and kidney tissue. Linear discriminant analysis for swine tissues. Cross validation of 100% achieved. Model developed using Na, Mn, Mg, P, S, K, Mo, Ca, Se, Zn, Fe, Si, Tl and Hf in order of importance.

FIG. 24: Multiplication factors for the normalisation of trace element concentration in tongue to the respective concentration in muscle tissue for swine. The variable line denotes the multiplication factor for tongue and the constant line denotes no change in element concentration between muscle and the tissue.

FIG. 25: Multiplication factors for the normalisation of trace element concentration in stomach to the respective concentration in muscle tissue for swine. The variable line denotes the multiplication factor for stomach and the constant line denotes no change in element concentration between muscle and the tissue.

FIG. 26: Multiplication factors for the normalisation of trace element concentration in heart to the respective concentration in muscle tissue for swine. The variable line denotes the multiplication factor for heart and the constant line denotes no change in element concentration between muscle and the tissue.

FIG. 27: Multiplication factors for the normalisation of trace element concentration in liver to the respective concentration in muscle tissue for swine. The variable line denotes the multiplication factor for liver and the constant line denotes no change in element concentration between muscle and the tissue.

FIG. 28: Multiplication factors for the normalisation of trace element concentration in kidney to the respective concentration in muscle tissue for swine. The variable line denotes the multiplication factor for kidney and the constant line denotes no change in element concentration between muscle and the tissue.

FIG. 29: Linear discriminant analysis for swine muscle (A, white arrow), tongue (B), stomach (C), heart (D), liver (E) and kidney (F). Cross validation of 73.39% achieved. Model developed using Th, Zn, Cd, Ti, S, Mg, P, Hg, Nb, Ru, Fe, Lu, Dy, V, Te, Ce, Ag, Si, Sn, Na, Se, Ge, Tl, Sr, Mo, Zr, Ga, Tb, Eu, Ni, Ta, K, Ca, Nd, Sm, In, La, Yb, Ho, Gd, Pb, Pr, Y, Bi, Ba, Hg, Li and Sc in order of importance.

FIG. 30: Region of origin prediction to North-Eastern Australia (A), South-Eastern Australia (B) and Western Australia (C) for non-corrected multi-elemental signatures of edible swine tongue, stomach, heart, liver and kidney (open squares). Correct prediction of 35% achieved. Model developed using a muscle-specific data base with a cross validation of 100% achieved. Elements used were Rb, Se, As, W, Co, Mg, V, Hf, K, Pd, Mn, Nb, Ce, Ti, Zn, Zr, Er, Sb and U in order of importance.

FIG. 31: Region of origin prediction to North-Eastern Australia, South-Eastern Australia and Western Australia for factor normalised multi-elemental signatures of edible swine tongue, stomach, heart, liver and kidney. Correct prediction of 85% achieved. Model developed using a muscle-specific data base with a cross validation of 100% achieved. Elements used were Rb, Se, As, W, Co, Mg, V, Hf, K, Pd, Mn, Nb, Ce, Ti, Zn, Zr, Er, Sb and U in order of importance.

FIG. 32: Farm of origin prediction to Western Australian Farm 3 (WA F3) for swine tongue, stomach and heart samples (open squares). Correct prediction of 29% achieved. Model developed using a muscle-specific data base with a cross validation of 89.29% achieved. Elements used were Cs, Pb, Na, Hf, U, Sn, Sm, Mg, Rb, S, V, Bi, Th, Sc, Co and Tb in order of importance.

FIG. 33: Farm of origin prediction to WA F3 for swine tongue, stomach and heart samples (open squares). Correct prediction of 68% achieved. Model developed using a muscle-specific data base with a cross validation of 89.29% achieved. Elements used were Cs, Pb, Na, Hf, U, Sn, Sm, Mg, Rb, S, V, Bi, Th, Sc, Co and Tb in order of importance.

FIG. 34: Farm of origin assignment of a muscle sample from Farm BBB using the Physi-Trace™ database. Cross validation of 67.16% achieved. Major elements used to determine the model were Tl, Rb, Ge, Hg, Cs, Co, As, P, S, Ti, Sr, Na, Se and Zn in order of importance.

FIG. 35: Discriminant plot detailing separation of data pertaining to fresh and processed meat samples from Australia and Canada.

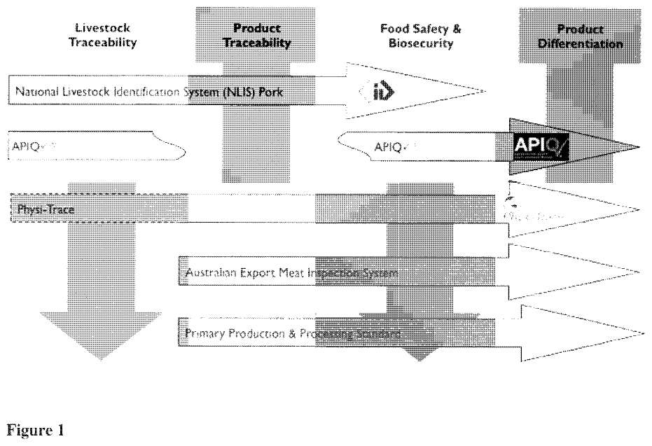

### DETAILED DESCRIPTION

The invention will now be described more particularly with reference to non-limiting examples.

Disclosed herein is a method of reporting the identity of an unknown pig sample, the method comprising:
a) registering a plurality of samples referenced to an individual pig animal or a group of pig animals in a database;
b) recording data representing the plurality of reference samples against the register;
c) recording data representing an unknown pig sample in the database;
d) comparing data representing the unknown pig sample with the data representing the plurality of reference samples to thereby assess the identity of the unknown pig sample through the register; and
e) generating a report providing an assessment of the identity of the unknown pig sample.

Sample Identity

Preferably, the identity is selected from the country of origin, the region of origin, the state of origin, the producer, the processor, or the property of origin. The property of origin is preferably linked to a tattoo, property identification code (PIC) and/or Australian Pork Limited (APL) PigPass Registration Number. Preferably, the identity is the individual pig animal.

Sample Types

Preferably, the plurality of samples referenced to an individual pig animal or a group of pig animals is a plurality of reference samples taken from pig tissue. Preferably, the pig tissue is muscle. The pig tissue is preferably raw tissue. The raw tissue is preferably muscle. Preferably, the muscle is selected from abdominal muscle. The abdominal muscle is preferably the transversalis muscle. Preferably, the pig tissue is offal. The offal is preferably selected from tongue, stomach, heart, liver, or kidney. Preferably, the tongue tissue is taken from the verticalis muscle, the transversalis muscle or the genioglossus muscle. Preferably, the stomach tissue is tissue taken from the corpus, the fundus or the pyloric antrum. The heart tissue is preferably tissue taken from the left ventricular wall, the right ventricular wall, the intraventricular septum, the superior ventricular wall, or the left atrial wall. Preferably, the liver tissue is tissue taken from the caudate lobe. Preferably the liver tissue is tissue taken from the caudate lobe excluding any veins, arteries, fatty tissue and/or connective tissue. The kidney tissue is preferably tissue taken from the renal cortex or renal pyramid. Preferably, the pig tissue is hair.

The plurality of samples referenced to an individual pig animal or a group of pig animals is preferably taken from a pork product. Preferably, the pork product is a processed pork product. The processed pork product is preferably selected from whole muscle bacon or ham. Preferably, the pork product is a comminuted product. The comminuted product is preferably selected from salami or sausage.

Sample Collection and Sample Collection Rules

The method preferably further comprises a sampling protocol for determining the number of samples to be taken for analysis. Preferably, the sampling protocol is based on the number of pigs killed. Preferably, the sampling protocol is based on the number of pigs killed in a given week at an abattoir. The sampling protocol is preferably based on the number of pigs killed in a given week at an abattoir and the number of unique tattoos that appear in the given week. Preferably, the sampling protocol comprises totaling the number of pigs for a given tattoo code killed in the given week.

If the total number of pigs for a given tattoo code killed in the given week is 1000 or greater, then preferably about ten to about twenty samples are taken. Preferably, about twenty samples are taken. About fifteen samples are preferably taken. Preferably, ten samples are taken.

Preferably, if the total number of pigs for a given tattoo killed in the given week is between 100 and 999, then about 5 to about twenty samples are taken. Preferably, about twenty samples are taken. About fifteen samples are preferably taken. Preferably, ten samples are taken. Five samples are preferably taken.

If the total number of pigs for a given tattoo killed in the given week is between 30 and 99, then preferably 3 samples are taken.

Preferably, if the total number of pigs for a given tattoo killed in the given week total is less than 30, then no samples are taken.

Preferably, a sample of about 5 g to about 100 g is collected. A sample of about 5 g to about 10 g is preferably collected. About 0.1 to about 5% of all samples taken are preferably randomly selected and submitted for analysis. Preferably, about 0.1 to less than about 5%; about 0.1 to less than about 4%; about 0.1 to less than about 3%; about 0.1 to less than about 2%; or about 0.1 to less than about 1% of all samples taken are randomly selected and submitted for analysis.

Wherein the foodstuff is selected from whole muscle bacon or ham, the method further preferably comprises a sampling protocol based on the number of ham or bacon samples sourced from the country of origin per month. Preferably, the country of origin is Australia. Preferably, a sample of about 10 to 50 g is collected. Preferably, a sample of about 40 g is collected. A sample of about 30 g is preferably collected. Preferably, a sample of about 20 g is collected. A sample of about 10 g is preferably collected. Preferably, about 5 to about 10% of ham or bacon samples sourced from the country or origin are submitted for analysis. Preferably, the country of origin is Australia. Preferably, about 5 to less than about 10%; about 5 to less than about 9%; about 5 to less than about 8%; about 5 to less than about 7%; or about 5 to less than about 6% of ham or bacon samples sourced from the country of origin are submitted for analysis. Preferably, the country of origin is Australia. The method preferably further comprises a sampling protocol where no less than 5 ham and 5 bacon samples are taken from product manufactured from pork sourced from within a region within a country other than the country of origin every month. Preferably, the country of origin is Australia. Preferably, the country other than Australia is selected from a group comprising: Canada, USA, Denmark, UK and the Netherlands. Preferably, a sample of about 20 g is collected. Preferably, about 5 to about 10% of all samples taken are submitted for analysis. Preferably, about 5 to less than about 10%; about 5 to less than about 9%; about 5 to less than about 8%; about 5 to less than about 7%; or about 5 to less than about 6% of all samples taken are submitted for analysis. Preferably, the 10% of samples are randomly sampled within the country of origin. Preferably, a section of about 5 g of the sample is sampled and submitted for analysis. The remainder of the sample is preferably returned to storage at about −18° C. for a period of 12 months. The sampling protocol preferably further comprises maintaining the samples submitted for analysis at about −18° C. prior to analysis.

Sample Registration

Preferably, the samples are registered in the database. The samples are preferably registered using a management system selected from a customer relationship management (CRM) system or a laboratory management system.

Sample Handling, Digestion and Analysis

Preferably, samples are analysed by a spectrometric and/or spectroscopic method. The samples are preferably analysed by a solution-based method. Preferably, the solution-based method is spectrometric and/or spectroscopic. The samples are preferably chemically digested to allow subsequent analysis. Preferably, the samples are thawed prior to chemical digestion. The samples are preferably allowed to thaw at room temperature. Preferably, sub-samples are taken for chemical digestion and subsequent analysis. The sub-samples are preferably about 2 g wet weight. Preferably, the sub-samples are taken so as to exclude any substantial fat. Excess moisture is preferably removed from the sub-samples. Preferably, the excess moisture is removed by placing the sub-samples on a paper towel for a period of about ten minutes. The wet weight of the sub-samples is preferably recorded. Preferably, dry weight analysis of the sub-samples is performed. The sub-samples are preferably chemically digested with a mixture of nitric acid and hydrogen peroxide. Preferably, the chemical digestion is carried out in sterile polypropylene tubes. The nitric acid is preferably quartz redistilled nitric acid.

Preferably, sub-samples of samples submitted for analysis are analysed by a spectrometric and/or spectroscopic method. Preferably, sub-samples of samples submitted for analysis are analysed by a solution-based method. Preferably, the solution-based method is spectrometric and/or spectroscopic. Preferably, the sub-samples of samples submitted for analysis are chemically digested to allow subsequent analysis. The sub-samples of samples submitted for analysis are preferably chemically digested with a mixture of nitric acid and hydrogen peroxide.

Digestion of Offal Samples

If the samples are offal, the chemical digestion preferably comprises the following steps:

adding nitric acid to the samples;

adding aqueous hydrogen peroxide to the nitric acid and sample mixtures;

cold digesting the sample mixtures for a time period until the samples have begun to break down; and

heating the sample mixtures at a sufficient temperature and for a sufficient time period to allow dissolution.

Preferably, a further amount of aqueous hydrogen peroxide may be added to the sample mixtures and heating continued.

The chemical digestions are preferably prepared for analysis by evaporation and then dilution with an appropriate solvent.

If the samples are offal, the chemical digestion preferably comprises the following steps:

a) adding 4 mL nitric acid to the polypropylene tubes containing the samples;

b) adding 2 mL 30% v/v aqueous hydrogen peroxide to the nitric acid sample mixtures and capping the tubes;

c) cold digesting the sample mixtures for about 24 hours to 48 hours until the samples have begun to break down;

d) heating the nitric acid:hydrogen peroxide sample mixtures to 60 to 70° C.;

e) heating the sample mixtures under reflux for about 12 hours;

f) increasing the temperature to 95 to 105° C. and removing the caps of the tubes;

g) adding a further amount of 2 mL 30% v/v aqueous hydrogen peroxide to the sample mixtures and capping the tubes; and

h) further heating the sample mixtures under reflux for about 2 hours.

Preferably, for steps d) to h) the sterile polypropylene tubes are placed in a water bath. The nitric acid is preferably quartz redistilled nitric acid.

Preferably, the chemical digestions are prepared for analysis by the following steps:

a) removing the caps of the tubes and allowing the digestion solutions to evaporate down to 2 mL;

b) making the digestion solutions up to 30 mL with deionised water; and

c) diluting the digestion solutions by a factor of about five-fold with a 2% v/v nitric acid solution containing an internal standard for monitoring analytical drift.

The 2% v/v nitric acid solution preferably contains Rh and Ir as standards for monitoring analytical drift. Preferably, the 2% v/v nitric acid solution contains Rh and Ir at a concentration of 2 μg/L.

Digestion of Pork Product Samples

If the samples are taken from a pork product, wherein the product is a processed product or a comminuted product as described herein, the chemical digestion preferably comprises the following:

digesting the samples in an aqueous mixture of nitric acid and hydrogen peroxide;

heating the nitric acid:hydrogen peroxide sample mixtures to allow dissolution; and

heating for evaporation.

Preferably, the chemical digestions are prepared for analysis by dilution with an appropriate solvent.

If the samples are ham or bacon, the chemical digestion preferably comprises the following steps:

a) digesting the samples in 5:2 nitric acid:hydrogen peroxide by volume and heating at 50° C. overnight; and

b) increasing the temperature to 90° C. for evaporation.

The nitric acid is preferably quartz redistilled nitric acid.

The chemical digestion is preferably prepared for analysis by dissolution in deionised water.

Digestion of Muscle Samples

If the samples are muscle, the chemical digestion preferably comprises the following steps:

adding nitric acid to the samples;

heating the nitric acid sample mixtures to allow dissolution;

evaporation;

addition of aqueous hydrogen peroxide.

Preferably, if the samples are muscle, the chemical digestion comprises the following steps:

adding nitric acid to the samples;

heating the nitric acid sample mixtures to allow dissolution;

evaporation;

adding nitric acid;

evaporation; and

addition of aqueous hydrogen peroxide.

If the samples are muscle, the chemical digestion preferably comprises the following steps:

a) setting the polypropylene tubes containing the samples at 90° C.;

b) adding 4 mL nitric acid to the tubes and capping the tubes;

c) holding the nitric acid sample mixtures at 90° C. for about 8 to 12 hours until the samples have dissolved;

d) removing the caps of the tubes and allowing the digestion solutions to evaporate to near dryness;

e) adding a further amount of 2 mL nitric acid and allowing the resulting solutions to evaporate at 90° C. until 1 mL of solution remains;

f) allowing the solutions to cool to 50° C. and then adding 1 mL hydrogen peroxide;

g) adding a further amount of 1 mL hydrogen peroxide after the initial peroxide reaction subsides; and

h) allowing the solutions to cool to room temperature after the further peroxide reaction subsides.

Preferably, the chemical digestions are prepared for analysis by making the digestion solutions up to about 40 mL, by mass, with deionised water. Preferably, for steps a) to e) the polypropylene tubes are placed in a water bath set at about 90° C.

The nitric acid is preferably quartz redistilled nitric acid.

Sample Analysis

Preferably, the samples are analysed for metals and selected non-metal elements. Preferably, the sub-samples of samples submitted for analysis are analysed for metals and selected non-metal elements. The metals and selected non-metal elements are preferably selected from the group consisting of:
sodium, magnesium, silicon, phosphorus, sulphur, potassium, calcium, manganese, iron, copper, zinc, aluminium, scandium, lithium, beryllium, boron, titanium, vanadium, chromium, cobalt, nickel, gallium, germanium, arsenic, selenium, rubidium, strontium, yttrium, zirconium, niobium, molybdenum, ruthenium, rhodium, palladium, silver, cadmium, indium, tin, antimony, tellurium, caesium, barium, lanthanum, cerium, praseodymium, neodymium, samarium, europium, gadolinium, terbium, dysprosium, holmium, erbium, thulium, ytterbium, lutetium, hafnium, tantalum, tungsten, gold, rhenium, iridium, platinum, mercury, thallium, lead, bismuth, thorium, and uranium.

Preferably, the metals and selected non-metal elements are analysed by Inductively Coupled Plasma Atomic Emission Spectrophotometry (ICP-AES) with the exception of lanthanum, cerium, praseodymium, neodymium, samarium, europium, gadolinium, terbium, dysprosium, holmium, erbium, thulium, ytterbium and lutetium.

Preferably, sodium, magnesium, silicon, phosphorus, sulphur, potassium, calcium, manganese, iron, copper, zinc, aluminium and scandium are analysed by ICP-AES. Sodium, magnesium, silicon, phosphorus, sulphur, potassium, calcium, manganese, iron, copper, zinc, aluminium and scandium are preferably respectively analysed at 589.5, 279.5, 251.6, 178.2, 180.7, 766.4, 422.6, 257.6, 239.5, 324.7, 213.8, 167.0 and 361.4 nm.

Preferably, lithium (Li), beryllium (Be), boron (B), aluminium (Al), scandium (Sc), titanium (Ti), vanadium (V), chromium (Cr), cobalt (Co), nickel (Ni), copper (Cu), zinc (Zn), gallium (Ga), germanium (Ge), arsenic (As), selenium (Se), rubidium (Rb), strontium (Sr), yttrium (Y), zirconium (Zr), niobium (Nb), molybdenum (Mo), ruthenium (Ru), palladium (Pd), silver (Ag), cadmium (Cd), indium (In), tin (Sn), antimony (Sb), tellurium (Te), caesium (Cs), barium (Ba), lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), tantalum (Ta), tungsten (W), mercury (Hg), thallium (Tl), lead (Pb), bismuth (Bi), thorium (Th) and uranium (U) are analysed by Inductively Coupled Plasma Mass Spectrometry (ICP-MS).

Preferably, ⁷Li, ⁹Be, ¹¹B, ²⁷Al, ⁴⁵Sc, ⁴⁹Ti, ⁵¹V, ⁵³Cr, ⁵⁹Co, ⁶⁰Ni, ⁶⁵Cu, ⁶⁶Zn, ⁶⁹Ga, ⁷¹Ga, ⁷²Ge, ⁷⁴Ge, ⁷⁵As, ⁸²Se, ⁸⁵Rb, ⁸⁸Sr, ⁹⁸Y, ⁹⁰Zr, ⁹³Nb, ⁹⁸Mo, ¹⁰¹Ru, ¹⁰³Rh, ¹⁰⁵Pd, ¹³⁶Pd, ¹⁰⁷Ag, ¹⁰⁹Ag, ¹¹¹Cd, ¹¹⁵In, ¹¹⁸Sn, ¹²⁰Sn, ¹²¹Sb, ¹²³Sb, ¹²⁶Te, ¹³³Cs, ¹³⁷Ba, ¹³⁸Ba, ¹³⁹La, ¹⁴⁰Ce, ¹⁴¹Pr, ¹⁴⁶Nd, ¹⁴⁷Sm, ¹⁵²Sm, ¹⁵³Eu, ¹⁵⁷Gd, ¹⁵⁸Gd, ¹⁵⁹Tb, ¹⁶²Dy, ¹⁶³Dy, ¹⁶⁵Ho, ¹⁶⁶Er, ¹⁶⁹Tm, ¹⁷²Yb, ¹⁷⁵Lu, ¹⁷⁸Hf, ¹⁸¹Ta, ¹⁸²W, ¹⁸⁵Re, ¹⁹³Ir, ¹⁹⁵Pt, ¹⁹⁷Au, ²⁰²Hg, ²⁰⁵Tl, ²⁰⁸Pb, ²⁰⁹Bi, ²³²Th and ²³⁸U are analysed by ICP-MS.

Preferably, vanadium (⁵¹V), chromium (⁵³Cr), and arsenic (⁷⁵As) are analysed by Inductively Coupled Plasma Collision Cell Mass Spectrometry (ICP-CC-MS). It will be appreciated analysis is preferably monitored to correct for instrumental drift.

Table 1 summarises the elements analysed for the particular instrumentation used.

TABLE 1
Elements and their relevant wavelengths (nm) or isotope
mass (m/z) analysed usingICP-AES, ICP-MS or ICP-CC-MS
for the solution based analysis of pig samples.

| Instrumentation | Elements analysed |
|---|---|
| ICP-AES | Na^{589.5}, Mg^{279.5}, Si^{251.6}, P^{178.2}, S^{180.7}, K^{766.4}, |
| | Ca^{422.6}, Mn^{257.6}, Fe^{239.5},Cu^{324.7}, Zn^{213.8}, Al^{167.0}, |
| | Sc^{361.4} |
| ICP-IVIS | ⁷Li, ⁹Be, ¹¹B, ²⁷Al, ⁴⁵Sc, ⁴⁹Ti, ⁵¹V, ⁵³Cr, ⁵⁹Co, |
| | ⁶⁶Ni, ⁶⁵Cu, ⁶⁶Zn, ⁶⁹Ga, ⁷¹Ga, ⁷²Ge, ⁷⁴Ge, ⁷⁵As, |
| | ⁸²Se, ⁸⁵Rb, ⁸⁸Sr,⁹⁸Y, ⁹⁰Zr, ⁹³Nb, ⁹⁸Mo, ¹⁰¹Ru, |
| | ¹⁰³Rh, ¹⁰⁵Pd, ¹⁰⁶Pd, ¹⁰⁷Ag, ¹⁰⁹Ag, ¹¹¹Cd, ¹¹⁵In, |
| | ¹¹⁸Sn, ¹²⁰Sn,¹²¹Sb, ¹²³Sb, ¹²⁶Te, ¹³³Cs, ¹³⁷Ba, |
| | ¹³⁸Ba, ¹³⁹La, ¹⁴⁰Ce, ¹⁴¹Pr, |
| | ¹⁴⁶Nd, ¹⁴⁷Sm, ¹⁵²Sm, ¹⁵³Eu, ¹⁵⁷Gd, ¹⁵⁸Gd, |
| | ¹⁵⁹Tb, ¹⁶²Dy, ¹⁶³Dy, ¹⁶⁵Ho, ¹⁶⁶Er, ¹⁶⁹Tm, ¹⁷²Yb, |
| | ¹⁷⁵Lu, ¹⁷⁸Hf, ¹⁸¹Ta, ¹⁸²W, ¹⁸⁵Re, ¹⁹³Ir, ¹⁹⁵Pt, |
| | ¹⁹⁷Au, ²⁰²Hg, ²⁰⁵Tl, ²⁰⁸Pb, ²⁰⁹Bi, ²³²Th, ²³⁸U |
| ICP-CC-MS | ⁵¹V, ⁵³Cr, ⁷⁵As |

Use of Standards in Analysis

Preferably, chemical digestion and analysis of the samples is run in parallel with at least one standard sample of known composition and known weight for assessing the quality of the analytical data. If the sample is offal, the chemical digestion and analysis is preferably run in parallel with a known weighed amount of National Institute of Standards and Technology (NIST) Bovine Liver 1577c Certified Reference Material (CRM) as a standard sample. Preferably, if the sample is muscle tissue, the chemical digestion and analysis is run in parallel with a known weighed amount of CRM DOLT 4, CRM DORM 3, and CRM TORT 2 (National Research Council of Canada) as standard samples. If the sample is a processed foodstuff, the chemical digestion and analysis is preferably run in parallel with a known weighed amount of CRM DOLT-4, CRM TORT-2 (National Research Council of Canada), and NCS DC73347 (China National Analysis Centre) as standard samples. The known amount is preferably between 0.2 to 0.5 g wet weight of the standard(s). Preferably, dry weight analysis of the standard(s) is performed. Preferably, for each analytical batch run fifteen percent of the samples are analysed in duplicate to determine reproducibility of the analytical data. Preferably, a minimum of three cross-over samples from a previous batch run are incorporated into the batch run to determine batch variation between analytical runs.

Preferably, chemical digestion and analysis of the sub-samples of samples submitted for analysis is run in parallel with at least one standard sample of known composition and known weight for assessing the quality of the analytical data. Preferably, for each analytical batch run fifteen percent of the sub-samples of samples submitted for analysis are analysed in duplicate to determine reproducibility of the analytical data.

Data Processing

The analytical data is preferably processed. Analytical data is preferably then recorded in the database. The raw output data file is preferably retained in the database when the analytical technique is ICP-MS. Preferably, software automatically retains the raw output data file in the database. The data file is preferably catalogued according to the date of sample analysis and/or the place of analysis. The place of analysis preferably is a testing laboratory. Preferably, the testing laboratory is an accredited testing laboratory. The data file is preferably accessible. The raw output data file is preferably copied. The copy of the data output file is preferably further processed. Preferably, the copy is implemented by software.

Data Quality Assurance and Correction

Preferably, processing the analytical data comprises processing the data for quality assurance. Processing the analytical data for quality assurance preferably comprises filtering the data. Preferably, filtering the analytical data comprises filtering the data to provide a completed concentration dataset. The processing preferably comprises the step of filtering ICP-MS analytical data to provide a completed ICP-MS concentration dataset. Preferably, the processing comprises the step of filtering ICP-CC-MS analytical data to provide a completed ICP-CC-MS concentration dataset. The processing preferably comprises the step of filtering ICP-AES analytical data to provide a completed ICP-AES concentration dataset.

Preferably, filtering analytical data comprises the step of recognition of missing or compromised data. The filtering further comprises the steps of:

adjustment of analytical data depending on counts per second (cps) recorded;

assessment of calibration standards;

correcting for interference from isobaric overlap and polyatomic interference;

identifying and correcting analytical trains;

drift correction;

blank correction;

concentration calculation from cps data;

calculation of detection limit and limit of determination;

removal of ancillary laboratory solutions and internal standards;

identification and selection of preferred isotopes when analytical technique is ICP-MS;

identification and selection of preferred wavelength when analytical technique is ICP-AES;

assessing the measured values for standard samples;

comparison of duplicate samples;

comparison of replicate samples;

comparison of crossover samples;

rechecking of in-house standards following crossover correction; and

cross comparison of ICP-AES, ICP-MS and/or ICP-CC-MS data.

Preferably, the standard samples are reference in-house standards and/or Certified Reference Materials standards. Preferably, the standard samples are reference in-house standards. The standard samples are preferably Certified Reference Materials standards.

Recognition of Missing or Compromised Sample Data

Preferably, the datasets are reviewed for missing data. Software preferably scans and determines if there is any data omitted and highlights these for assessment. Preferably, if the data is presented in a spreadsheet format, any cells without data are highlighted for assessment. The dataset is preferably reviewed for compromised data. Preferably, median values of pork samples, excluding ancillary samples, are calculated. Ancillary samples are preferably selected from: blanks, Certified Reference Materials, in-house standards, wash or drift solutions. Preferably, if the count rate for any samples is half or twice the median value, affected data cells are highlighted and presented to the analyst who will decide if the sample(s) is (are) included in subsequent correction procedures or whether it is removed from the dataset and re-analysed.

Data Adjustment of Values Less than 1 cps

Preferably, all analytical data which is recorded with a value of less than one cps is replaced with a value of 1.00 when the analytical technique is ICP-AES or ICP-MS.

Assessing the Calibration Standards

Preferably, calibration curves are plotted for each element and a linear trend line fitted. The co-efficient of determination R² is calculated for each calibration curve. The analyst can view each calibration curve if required. All potentially erroneous calibration curves with an R² of less than 0.985 will be identified and reviewed by the analyst. Individual erroneous standards can be removed and R² recalculated. If all standards of a given element are erroneous, then all count values are set to zero and concentration data for this element will not be derived in any of the samples in the dataset.

Correcting for Isobaric Overlap and/or Polyatomic Interferences when Conducting ICP-MS

The ICP-MS analytical data is preferably corrected for errors associated with isobaric overlap and/or polyatomic interferences. Preferably, the correction comprises changing the dissolution method, instrumentation set-up, and/or mathematically accounting for the errors post analysis. The correction preferably comprises mathematically accounting for the errors post analysis. Preferably, changing the instrumentation set-up comprises altering ICP-MS operating parameters. Altering ICP-MS operating parameters preferably comprises using a reaction gas, a reaction cell or a collision cell to reduce polyatomic interferences.

Preferably, if a reaction gas or a reaction cell is used the following gases are usable for reducing polyatomic interferences: H₂, NH₃, Xe, CH₄, N₂O, NO, CO₂, CO, C₂H₆, C₂H₄, CH₃F, SF₆, CH₃OH or a mixture thereof. In one embodiment, if a collision cell is used inert gases are usable for reducing polyatomic interferences. Preferably, the inert gases are selected from He, Ar, Ne, Xe or a mixture thereof.

Correcting the Isobaric Overlap Interference on Arsenic (⁷⁵As) when Conducting ICP-MS

ICP-MS analytical data is, for example, corrected for errors associated with isobaric overlap. For illustration, a correction for ⁷⁵As is undertaken due to the ⁴⁰Ar³⁵Cl interference present on the same mass. The correction is performed by using measured values of the krypton isotopes (⁸²Kr and ⁸³Kr) and selenium isotopes (⁷⁷Se and ⁸²Se) to determine the extent of the interference on mass 75, and then calculating the “true” count rate that is attributable to arsenic. The “true” count rate calculated for all samples replaces the measured ⁷⁵As count rates recorded by the ICP-MS instrument.

Thus, according to the method of reporting the identity of an unknown pig sample disclosed herein, correcting for errors associated with isobaric overlap preferably comprises correcting the ICP-MS analytical data for the interference on arsenic (⁷⁵As). Preferably, correcting the interference on arsenic (⁷⁵As) comprises correcting for the ⁴⁰Ar³⁵Cl interference. Correcting for the ⁴⁰Ar³⁵Cl interference preferably comprises the following steps:

a) measuring the count rate value of the krypton isotope (⁸³Kr) to remove the krypton contribution on the ⁸²Se and ⁸²Kr signal to determine a count rate of ⁸²Se;

b) using the values of the selenium isotopes (⁷⁷Se and ⁸²Se) to determine the extent of the interference on mass 75;

c) calculating a count rate that is attributable to ⁷⁵As; and

d) replacing the measured count rates at mass 75 recorded by the ICP-MS instrument with the calculated count rate that is attributable to ⁷⁵As.

Identifying and Correcting Analytical Trains

XY Scatter Plots representing the measured count rates of an analyte in the first 20 samples are constructed for those analytes commonly found to have analytical trains (19 from a complete list of 97 analytes). The analyst is asked for each to confirm or deny the presence of an analytical train based on the presentation of the two graphs. For data with an analytical train, a number of calculations are undertaken to mathematically describe the form of the analytical train (a power curve), the parameters of which dictate the correction to be applied to the raw data. The “true” count rates calculated for the first 20 samples of the affected analyte replace the measured values recorded by the ICP-MS instrument.

Drift Correction

In order to account for any instrumental drift (the change in instrument sensitivity as the analytical run progresses), three internal standards are used when running ICP-MS: beryllium (⁹Be, for analytes ⁷Li to ⁶⁶Zn), rhodium (¹⁰³Rh, for analytes ⁶⁹Ga to ¹³⁸Ba) and iridium (¹⁹¹Ir and ¹⁹³Ir, for analytes ¹³⁹La to ²³⁸U). All samples are normalized relative to one another to correct for any instrumental drift that has occurred. When running ICP-AES a drift solution made up of a wash of 5% v/v HNO₃ and 18 MegΩ MilliQ deionised water is used.

Blank Correction

As the pork solutions represent pork meat dissolved into an acidic aqueous solution, the measured count rate of a particular element in a sample will represent the actual (true) amount in both the sample and any reagents used to prepare the solution. Blank samples are used to determine the contribution of the reagents and therefore, the true amount in the sample. For each element, the median count rate is determined from no less than three blank samples and this value is subtracted from the measured count rate for all samples (including Certified Reference Materials and in-house standards). In the event that blank samples record values greater than that found in samples (which would result in negative count rates and therefore negative concentration in a sample, both of which are chemical impossibilities), the median value will be determined in a different manner.

Concentration Calculation (ppb) or (ppm) from CPS Data

Using approved calibration standards for analytes, the ICP-MS CPS data is transformed into ppb data and the ICP-AES CPS data is transformed into ppm data.

Calculation of Detection Limits and Limits of Determination

Using the 0 ppb solutions for ICP-MS or the 0 ppm solutions for ICP-AES the detection limit and limit of determination for the dataset are determined. Preferably, all analytical data which is recorded as less than detection limit (<DL) is replaced with 0.

Removal of Ancillary Laboratory Solutions and Internal Standards

All ancillary solutions (laboratory control samples that do not represent pork standards or pork samples acquired from abattoirs) are removed from the dataset. This includes blank sample solutions, HNO₃ and deionized water wash solutions, calibration standards and drift solution samples. In the case of ICP-MS, internal standards that are used for drift correction are removed. For example, internal standards beryllium (⁹Be), rhodium (¹⁰³Rh), and iridium (¹⁹¹Ir and ¹⁹³Ir) are removed as these elements are added in known quantities to allow drift correction to be undertaken.

Identification and Selection of Preferred Isotopes

In the instances where more than one isotope of a given element is measured using ICP-MS, procedures are undertaken to identify the most relevant isotope and to remove any additional isotope of that element.

Identification and Selection of Preferred Wavelength

In the instances where more than one wavelength of a given element is measured using ICP-AES, procedures are undertaken to identify the most relevant wavelength and to remove any additional isotope of that element.

Assessing the Measured Values for the in-House Reference Standards

Known values for the in-house reference standards (e.g. reference standards for pork liver, pork muscle, bacon, ham) are compared to the measured values for these standards. Any elements in each of these standards that fall outside the range specified are highlighted for consideration.

Comparison of Duplicate Samples (within-Dataset)

Comparison of the duplicate pairs is undertaken and the most reliable sample in the duplicate pair selected.

Comparison of Replicate Samples (within-Dataset) (10 Samples Analysed Start/End)

Comparison of the ten samples analysed at both the start and end of the analytical run. This comparison serves to indicate whether the analytical train correction has been successful. If this is the case, the ten samples analysed at the start of the analytical run are selected and those replicates analysed at the end of the analytical run are discarded. If this is not the case, the reverse process is undertaken.

Comparison of Crossover Samples (Between Datasets)

Comparison of crossover samples analysed on two separate occasions by the testing laboratory.

Calculation of any batch to batch differences is undertaken and mathematical corrections are applied to eliminate any discrepancies.

Assessing the Measured Values for the Certified Reference Materials

The measured values for NIST standard 1577c (Bovine Liver) and the in-house standards (eg reference standards for pork liver, pork muscle, bacon, ham) will be compared to the certified/known values to determine the accuracy of the method. If measured values fall outside the acceptable range of the certified values, a correction will be undertaken and applied to all samples (excluding drift solutions and instrument wash/rinse solutions).

Re-Checking of in-House Standards Following Crossover Correction

Following crossover correction, the in-house standards are rechecked to ensure that the measured elements fall within the known ranges specified. At this point, all corrections have been undertaken and if elements still fall outside the specified range, the analyst is required to make a decision about whether the analyte is accepted or rejected.

Preparation and Merging of ICP-AES, ICP-CC-MS and ICP-MS Data

Preferably, ICP-AES data is transformed into ppb units. A completed ICP-AES concentration dataset with metadata is preferably imported into the ICP-MS module and merged with existing ICP-MS sample data and/or ICP-CC-MS sample data. Preferably, sample data is metadata comprising identifying information. The identifying information is preferably selected from the group comprising: a laboratory code, batch number and sample identification code.

Cross Comparison of ICP-AES, ICP-CC-MS and ICP-MS Data

For elements analysed by both ICP-MS and ICP-AES, a comparison is made between the two values calculated by both software modules. A determination is made by the analyst as to which value to select and incorporate into the final dataset. For elements analysed by ICP-AES, ICP-CC-MS and/or ICP-MS (for example, aluminium, scandium, copper and zinc), a comparison is made between the values calculated by the different software modules. For example, ⁵¹V, ⁵³Cr and ⁷⁵As can be analysed using either ICP-MS or ICP-CC-MS. A determination is made regarding the value to select and incorporate in the final dataset.

Data Export

Preferably, the final completed concentration dataset is transmitted to the database. The transmitting is preferably achieved by exporting the final completed concentration dataset to the database. Preferably, the final completed concentration dataset is exported to the database with metadata. The metadata is preferably identifying information. The identifying information is selected from the group comprising a laboratory code, batch number and sample identification code.

Data Interpretation

Preferably, the final completed concentration data is processed to provide data representing the plurality of reference samples. Preferably, the data representing the plurality of reference samples is in the form of a multi-elemental concentration profile representing the plurality of reference samples. The multi-elemental concentration profile is preferably presented as parts per billion (ppb) or parts per million (ppm) of each element based on the dry weight of the samples. The multi-elemental concentration profile is preferably presented as parts per billion (ppb) or parts per million (ppm) of each element based on the dry weight of the sub-samples of samples submitted for analysis. Preferably, a statistical tool is used to process the multi-elemental concentration profiles representing the plurality of reference samples. The statistical tool is preferably a multivariate statistical tool. Preferably, the multivariate statistical tool is selected from the group consisting of: linear discriminant analysis (LDA), principle component analysis (PCA), Wards method of hierarchical clustering, multinominal models (MN), support vector machines (SVM), mixture discriminate analysis (MDA), classification tree (CT) and neural networks (NN). The multivariate statistical tool is preferably LDA. Preferably, the LDA is conducted using a forward step-wise model. The model preferably has a tolerance level of 0.00001 and a significance level of 5%. The accuracy of the LDA model is preferably tested using a cross validation process. Preferably, the cross validation process comprises the comparison of raw analytical data for the samples. The comparison of raw analytical data preferably comprises the comparison of elemental associations. Preferably, the cross validation process is a leave-one-out cross validation. Preferably, processing the analytical data representing the plurality of reference samples further comprises standardising of multi-element concentration profiles for offal tissue data to multi-elemental concentration profiles for muscle tissue data to allow use of a single database for all raw pig tissues.

Preferably, the standardising comprises the calculation of multiplication factors that enable the normalisation of the chemical concentration in offal tissue back to muscle-equivalent concentrations. Processing the analytical data representing the plurality of reference samples preferably further comprises standardising of multi-elemental concentration profiles for processed foodstuff samples to multi-elemental concentration profiles for muscle tissue samples to allow use of a single database for all pig samples.

Tracing of Unknown Samples

Preferably, the unknown sample is taken from pig tissue. The pig tissue is preferably raw tissue. The raw tissue is preferably muscle. Preferably, the muscle is selected from abdominal muscle. The abdominal muscle is preferably the transversalis muscle. Preferably, the pig tissue is offal. The offal is preferably selected from tongue, stomach, heart, liver, or kidney. Preferably, the tongue tissue is taken from the verticalis muscle, the transversalis muscle or the genioglossus muscle. Preferably, the stomach tissue is tissue taken from the corpus, the fundus or the pyloric antrum. The heart tissue is preferably tissue taken from the left ventricular wall, the right ventricular wall, the intraventricular septum, the superior ventricular wall, or the left atrial wall. Preferably, the liver tissue is tissue taken from the caudate lobe. The tissue is preferably taken from the caudate lobe excluding any veins, arteries, fatty tissue and/or connective tissue. Preferably, the kidney tissue is tissue taken from the renal cortex or renal pyramid. The pig tissue is preferably hair.

The unknown pig sample is preferably taken from a pork foodstuff. Preferably, the pork foodstuff is a processed foodstuff. The processed foodstuff is preferably selected from whole muscle bacon or ham. Preferably, the pork foodstuff is a comminuted foodstuff. The comminuted foodstuff is selected from salami or sausage.

Preferably, the unknown pig sample is about 10 g. Sub-samples are preferably taken for the chemical digestion and analysis. Preferably, the sub-samples are about 2 g wet weight. The sub-samples are preferably taken so as to exclude any substantial fat. Preferably, excess moisture is removed from the sub-samples. The excess moisture is preferably removed by placing the sub-samples on a paper towel for a period of about ten minutes. The wet weight of the sub-samples is preferably recorded. Preferably, dry weight analysis of the sub-samples is performed. The sub-samples are preferably chemically digested with a mixture of nitric acid and hydrogen peroxide. Preferably, the chemical digestion is carried out in sterile polypropylene tubes.

Preferably, the chemical digestion comprises the following steps:

a) adding 5 mL nitric acid and 2 mL 30% hydrogen peroxide to the polypropylene tubes containing the samples;

b) capping the tubes; and

c) standing the nitric acid:hydrogen peroxide sample mixtures at 50° C. for about 9 hours.

Preferably, the chemical digestions are prepared for analysis by the following steps:

a) removing the caps of the tubes and setting the digestion solutions at 90° C. so that they evaporate down to 1 mL; and

b) making up the solutions to 30 mL with deionised water.

The digested sub-samples of the unknown pig sample are preferably analysed by the methods disclosed herein to provide data representing the unknown pig sample. Preferably, LDA is used in the step of comparing the data representing the unknown pig sample with the data representing the plurality of reference samples to thereby identify the unknown pig sample. Preferably, the step of comparing comprises integrating the data representing the unknown pig sample with the data representing the plurality of reference samples and conducting LDA using a forward step-wise model to thereby identify the unknown pig sample. The report can preferably be generated within about 24 hours of commencing digestion of a sub-sample of the unknown pig sample. Preferably, the method further comprises storing the report to the database.

The steps of:

(i) registering the plurality of reference samples in a database;

(ii) recording data representing the plurality of reference samples against the register; and/or

(iii) recording data representing the unknown pig sample in the database,

are preferably accomplished with a user interface in communication with the database with the proviso that the user has permission as set by an administrator.

Preferably, the report is provided to the user through the interface with the proviso that the user has permission to view the report, the permission being set by the administrator.

Also disclosed herein is a system for reporting the identity of an unknown pig sample, the system comprising:
(a) means for registering a plurality of samples referenced to an individual pig animal or a group of pig animals in a database;
(b) means for recording data representing the plurality of reference samples against the register;
(c) means for recording data representing the unknown pig sample;
(d) means for comparing data representing the unknown pig sample with the data representing the plurality of reference samples to thereby identify the unknown pig sample; and
(e) means for generating a report providing an assessment of the unknown pig sample.

Also disclosed herein is a computer implemented system for reporting the identity of an unknown pig sample, the computer implemented system comprising a processor configured to:
(a) register a plurality of samples referenced to an individual pig animal or a group of pig animals in a database;
(b) record data representing the plurality of reference samples against the register;
(c) record data representing the unknown pig sample;
(d) compare data representing the unknown pig sample with the data representing the plurality of reference samples to thereby assess the identify the unknown pig sample; and/or
(e) generate a report providing an assessment of the unknown pig sample.

Preferably, the processor is further configured to process the analytical data as disclosed herein.

Preferably, the processor is connected to a program memory, a data memory, a data port, and a database.

The program memory is preferably a non-transitory computer readable medium. Preferably, the non-transitory computer readable medium is selected from a hard drive, a solid state disk, DVD, USB drive or CD-ROM.

The data memory is preferably volatile memory or non-volatile memory. The volatile memory is preferably RAM or cache. The non-volatile memory is preferably selected from: ROM or a storage device. The storage device is preferably selected from a optical disk drive, hard disk drive, storage server or cloud storage. Preferably the data memory stores: data registering a plurality of samples referenced to an individual pig animal or a group of pig animals; data representing the plurality of reference samples against the register; data representing the unknown pig sample; information comparing data representing the unknown pig sample with the data representing the plurality of reference samples to thereby assess the identity of the unknown pig sample; and stores a report providing an assessment of the unknown pig sample. The data or information is preferably stored in a format selected from: CSV, TSV, look-up tables and graphs.

The dataport is preferably a port used to receive and transmit data. The data port is preferably a communications port or a user port. Preferably, the data port is a network connection, a memory interface, a pin of the chip package of the processor unit, or logical ports, such as IP sockets or parameters of functions stored on program memory and executed by the processor unit. The communications port preferably provides a plurality of communication links. Preferably, the links connect to one or more remote computing systems. The remote computer systems preferably are a remote server, personal computer, terminal, wireless or handheld computing device. Preferably, the links are hardwired, for example, via an Ethernet cable. The links preferably operate via a Wi-Fi network, 3G, the Internet or any combination thereof.

Preferably, the database is in communication with the remote computer system. Preferably, the database is part of the remote computer system. The database is preferably separate from the remote computer system. The database preferably resides on a disc or other storage device. Preferably, the database can be accessed by the processor unit to compare data representing the unknown pig sample with the data representing the plurality of reference samples to thereby assess the identity of the unknown pig sample. The database is preferably a customer relationship management (CRM) database. Preferably, the database is administered.

Preferably the database stores: data registering a plurality of samples referenced to an individual pig animal or a group of pig animals; data representing the plurality of reference samples against the register; data representing the unknown pig sample; information comparing data representing the unknown pig sample with the data representing the plurality of reference samples to thereby assess the identity of the unknown pig sample; and stores a report providing an assessment of the unknown pig sample. The data or information is preferably stored in a format selected from: CSV, TSV, look-up tables and graphs.

Preferably, the processor comprises:
(a) a registration module configured to register a plurality of samples referenced to an individual pig animal or a group of pig animals in the database;
(b) a reference module configured to record data representing the plurality of reference samples against the register;
(c) a sample module configured to record data representing the unknown pig sample;
(d) an interrogation module configured to compare data representing the unknown pig sample with the data representing the plurality of reference samples to thereby identify the unknown pig sample; and/or
(e) a report module configured to generate a report providing an assessment of the unknown pig sample.

Preferably, any receiving of analytical data may be preceded by a step wherein the analytical data is processed and then received. The processing preferably comprises processing the analytical data for quality assurance. Preferably, the processing comprises filtering the analytical data as disclosed herein. Thus the processor preferably further comprises a quality assurance module for filtering analytical data.

Preferably, the modules are implemented as hardware or software or a combination thereof. The modules are preferably separate from each other. Preferably, the modules are combined. Preferably, the modules are arranged to communicate with each other. The modules are preferably arranged to communicate with each other and the processor unit. The modules are preferably integrated with each other as a single hardware package. Preferably, the modules are integrated with each other as a single software package. The package preferably has the functionality of all the modules. The modules are preferably isolated from each other and in communication with each other even though they are part of a single package. The modules are preferably software modules within the processor or as software instructions stored on the program memory or the data memory.

The computer system is preferably implemented by a server or computer device having user interface means through which a user can communicate with the computer system. Preferably, the user interface means is a graphical user interface.

Preferably, the server is implemented by any appropriate computing architecture. The computing architecture is preferably a standalone PC, client/server architecture, terminal/mainframe architecture, or a laptop.

The computing device preferably provides interface means by which a user can input information into the computing system. Preferably, the computing device further comprises a display for displaying information to the user. The information is preferably the report providing the assessment of the identity of the unknown pig sample with the proviso that the user has permission to view the report, the permission being set by the administrator.

The computing system is appropriately programmed to perform a method of reporting the identity of an unknown pig sample as disclosed herein.

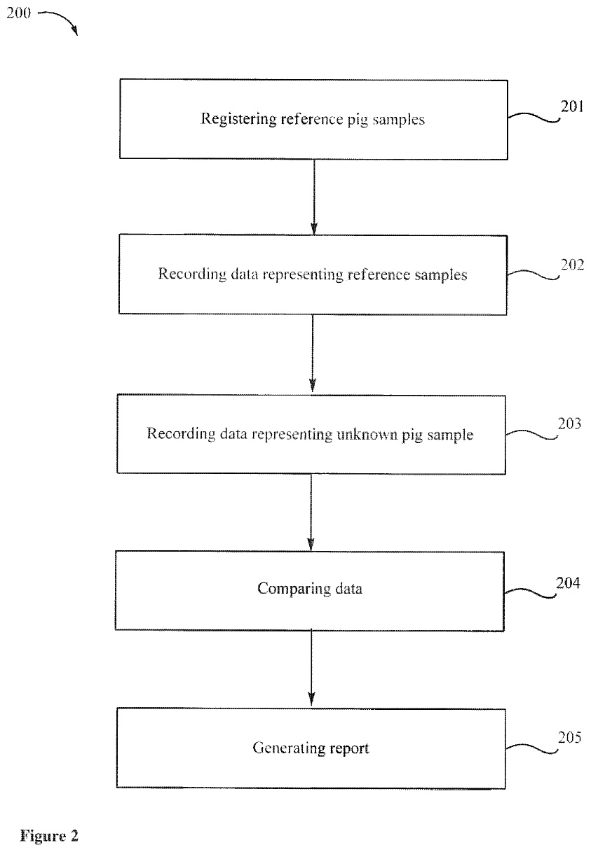

An example of a method of reporting the identity of an unknown pig sample will now be described with reference to FIG. 2.

The method starts with registering a plurality of samples referenced to an individual pig animal or a group of pig animals in a database (201). This step is followed by recording data representing the plurality of reference samples against the register (202) which precedes recording data representing the unknown pig sample in the database (203). The method continues with comparing data representing the unknown pig sample with the data representing the plurality of reference samples to thereby assess the identity of the unknown pig sample through the register (204). The method finishes with the step of generating a report providing an assessment of the identity of the unknown pig sample (205).

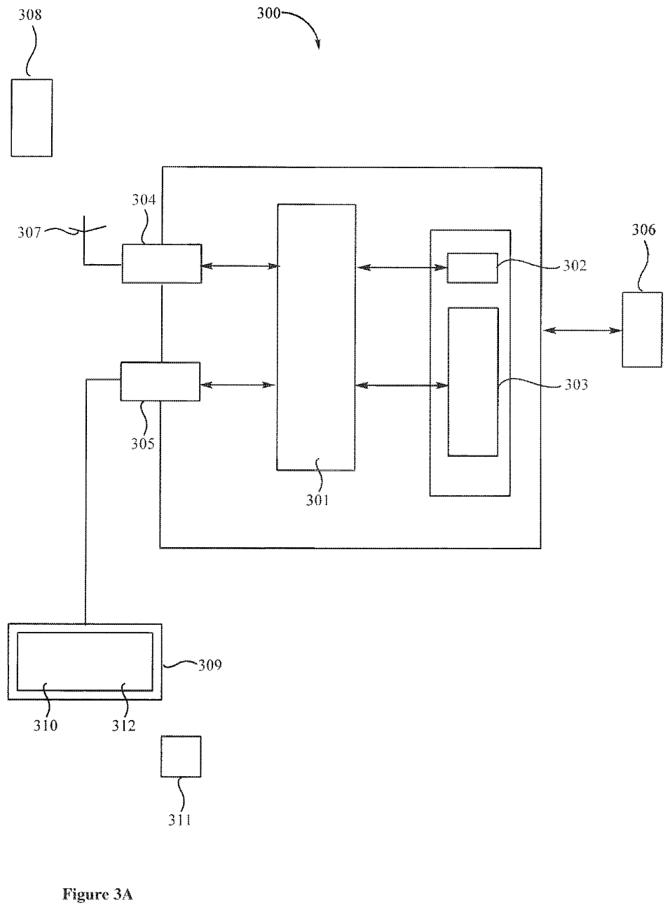

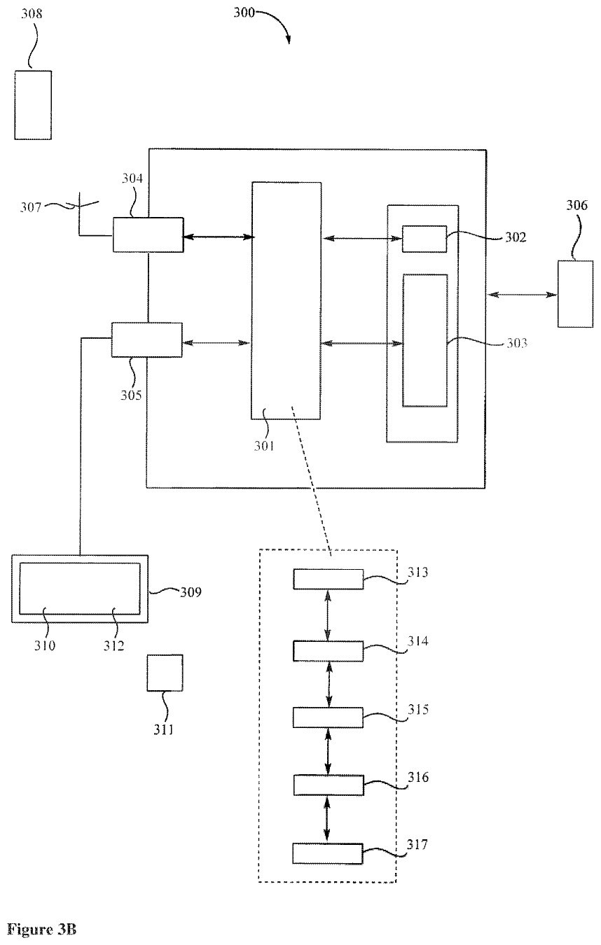

FIG. 3A depicts an example of a computer system for implementing the method of reporting the identity of an unknown pig sample shown in FIG. 2.

The computer system (300) comprises a processor (301) configured to:
(a) register a plurality of samples referenced to an individual pig animal or a group of pig animals in the database (306);
(b) record data representing the plurality of reference samples against the register;
(c) record data representing the unknown pig sample,
(d) compare data representing the unknown pig sample with the data representing the plurality of reference samples to thereby identify the unknown pig sample; and/or
(e) generate a report providing an assessment of the unknown pig sample.

The processor is further configured to process the analytical data as disclosed herein.

The processor 301 is connected to a program memory (302), a data memory (303), a data port (FIG. 3A refers to a communication port 304 and a user port 305), and a database (306).

The program memory 302 may be a non-transitory computer readable medium such as a hard drive, a solid state disk, DVD, USB drive or CD-ROM.

The data memory 303 may be volatile memory or non-volatile memory where the volatile memory may be RAM or cache and where the non-volatile memory may be ROM or a storage device. The storage device may be an optical disk drive, hard disk drive, storage server or cloud storage. The data memory 303 may store: data registering a plurality of samples referenced to an individual pig animal or a group of pig animals; store data representing the plurality of reference samples against the register; store data representing the unknown pig sample; store information comparing data representing the unknown pig sample with the data representing the plurality of reference samples to thereby identify the unknown pig sample; and store the report providing an assessment of the unknown pig sample. The data may be stored in, for example, CSV, TSV formats or, for example, as look-up tables or graphs.

The dataport may be any port that is used to receive and transmit data (FIG. 3A refers to the communication port 304 and the user port 305). Hence, it would recognised that a data port may a network connection, a memory interface, a pin of the chip package of the processor unit, or logical ports, such as IP sockets or parameters of functions stored on program memory and executed by the processor unit. The communication port 304 is designed to provide a plurality of communication links (307) which may connect to one or more remote computing systems, for example a server (308). The remote computer system may also be a personal computer, terminal, wireless or handheld computing device. The links may be hardwired, for example, via an Ethernet cable. The links may also operate via a Wi-Fi network, 3G, the Internet or any combination thereof.

The database 306 may be in communication with a computing device 309, as shown in FIG. 3A, or the server 308. The database may be part of the computer system as shown in FIG. 3A or separate. The database may reside on a disc or other storage device. The database may be a customer relationship management (CRM) database. It would be recognised that the database is administered according to an agreed set of business rules.

The database may store: data registering a plurality of samples referenced to an individual pig animal or a group of pig animals; data representing the plurality of reference samples against the register; data representing the unknown pig sample; information comparing data representing the unknown pig sample with the data representing the plurality of reference samples to thereby assess the identity of the unknown pig sample; and the report providing an assessment of the unknown pig sample. The data or information is may be stored in a format selected from: CSV, TSV, look-up tables and graphs.

The computer system may be implemented by a server, for example the server 308, or computer device, such as 309. The server or the computer device may have user interface means (FIG. 3A shows a user interface (310) for the computer device) through which a user (311) may communicate with the computer system. The user interface means may be in the form of a graphical user interface through which a user (311) may, for example, input information into the computing system.

The server may be implemented by any appropriate computing architecture, for example, a standalone PC, client/server architecture, terminal/mainframe architecture, or a laptop.

The computing device may further comprise a display (312) for displaying information to the user. The information may include the report providing the assessment of the identity of the unknown pig sample with the proviso that the user has permission to view the report, the permission set by the administrator.

In operation, software that is an executable program stored on the program memory or the data memory causes the processor to perform the method shown in FIG. 2, that is, the processor
(a) registers a plurality of samples referenced to an individual pig animal or a group of pig animals in a database;
(b) records data representing the plurality of reference samples against the register;
(c) records data representing the unknown pig sample,
(d) compares data representing the unknown pig sample with the data representing the plurality of reference samples to thereby identify the unknown pig sample; and
(e) generates a report providing an assessment of the unknown pig sample.

In operation, the processor may receive a request to register a plurality of samples referenced to an individual pig animal or a group of pig animals in the database 306 from the data memory 303 or from the communications port 304 and/or the user port 305, which are connected to the computer device 309 or server 308, which have a user interface 310 through which the user 316 may input the request. The processor may then record data representing the plurality of reference samples against the register and in turn data representing the unknown pig sample. Following this, the processor 301 may access, for example, the database 306 to compare data representing the unknown pig sample with the data representing the plurality of reference samples to thereby assess the identity of the unknown pig sample. The processor then generates the report providing an assessment of the unknown pig sample which is transmitted through the communication port 305 to, for example, the computer device 309 and then through the display 312 to the user 311 with the proviso that the user has permission to view the report, the permission set by the administrator.

It would be understood that any recording of data, such as analytical data, by the computer system may be preceded by a step wherein the data is processed. The processor may also be further configured to process the analytical data as disclosed herein. For example, the processor may be configured to process the analytical data for quality assurance as disclosed herein.

FIG. 3B depicts another example of a computer system for implementing the method of reporting the identity of an unknown pig sample in FIG. 2. The example of FIG. 3B is a variation of the example shown in FIG. 3A and like features are numbered the same. However, in comparison to the example computer system shown in FIG. 3A, the processor of the example computer system shown in FIG. 3B comprises:
(a) a registration module (313) configured to register a plurality of samples referenced to an individual pig animal or a group of pig animals in the database (306);
(b) a reference module (314) configured to record data representing the plurality of reference samples against the register;
(c) a sample module (315) configured to record data representing the unknown pig sample;
(d) an interrogation module (316) configured to compare data representing the unknown pig sample with the data representing the plurality of reference samples to thereby identify the unknown pig sample; and
(e) a report module (317) configured to generate a report providing an assessment of the unknown pig sample.

Any receiving of analytical data may be preceded by a step wherein the analytical data is processed and then received. The processing may comprise processing the analytical data for quality assurance such as filtering the analytical data. Thus, the processor may further comprise a quality assurance module for filtering analytical data as disclosed herein (the quality assurance module is not shown in FIG. 3B).

The modules may be implemented as hardware or software or a combination thereof. The modules preferably are implemented as separate software programs that can interact or interface with one each other. The modules may be implemented as separate hardware devices that can interact or interface with one each other. The modules may be combined or separate from each other. The modules may be arranged to communicate with each other and/or the processor unit.

The modules may be integrated with each other as a single hardware package or as a single software package.

The package may have the functionality of all the modules. The modules may be isolated from each other and in communication with each other even though they are part of a single package. As shown in FIG. 3B, the modules are located within the processor. The modules may be software instructions stored on the volatile or non-volatile memory of the system. The modules may be software instructions stored on the program memory of the system. The software instructions may cause the processor to perform the method of reporting the identity of an unknown pig sample shown in FIG. 2, that is, the processor:
(a) registers a plurality of samples referenced to an individual pig animal or a group of pig animals;
(b) records data representing the plurality of reference samples against the register;
(c) records data representing the unknown pig sample,
(d) compares data representing the unknown pig sample with the data representing the plurality of reference samples to thereby identify the unknown pig sample; and
(e) generates a report providing an assessment of the unknown pig sample.

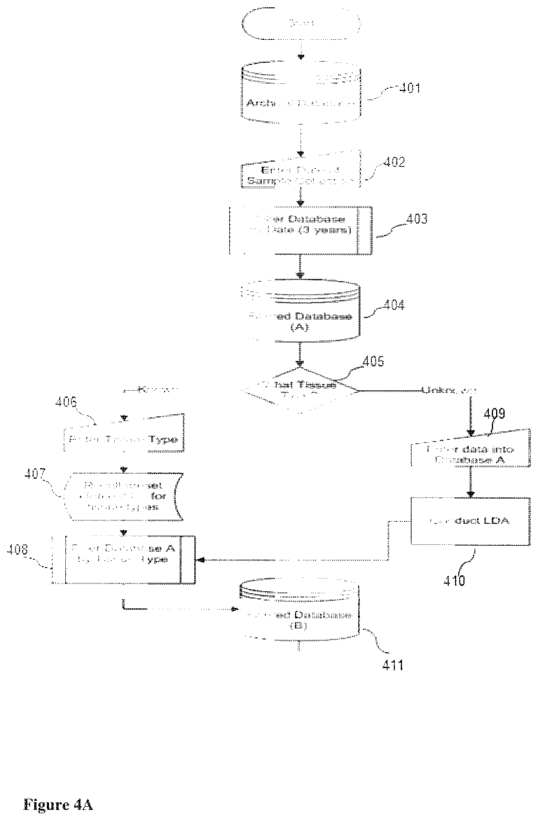

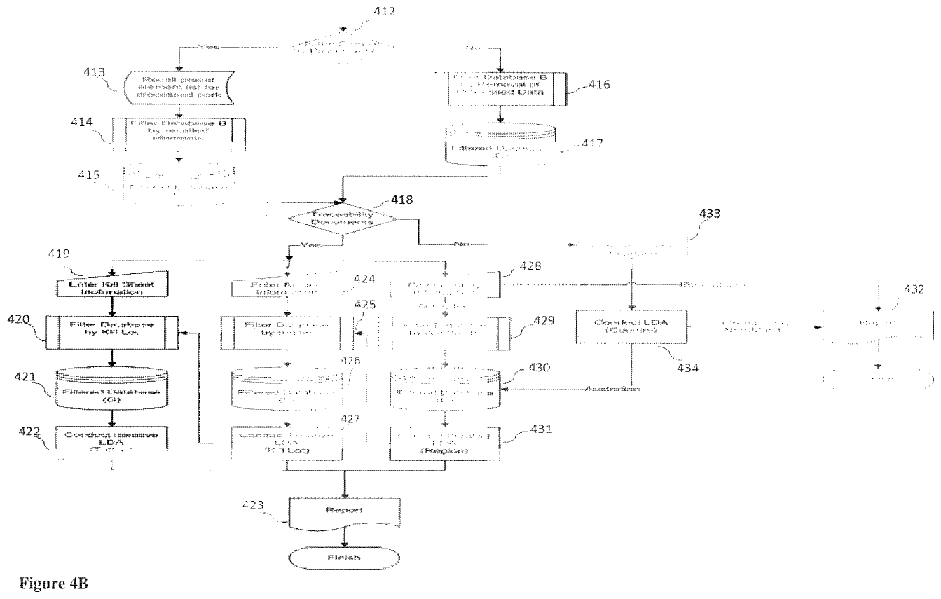

With reference to FIGS. 4A and 4B, the start point in the flow diagram, showing an algorithm for reporting the identity of an unknown pig sample, represents a decision to conduct a traceback. It would be appreciated that a traceback may be requested in a number of scenarios. The scenarios include:

Responding to a food safety event.

A consumer wishing to conduct a traceback on sample of meat.

A company wishing to a conduct a traceback of their own product to show an existing or potential customer.

A company wishing to a conduct a traceback of their product because they believe that substitution of the product has occurred, or an incident involving the product has occurred.

A government body wishes to conduct a traceback. For example, NRS (National Residue Survey) wants to do a traceback due to issues with a NRS sample that has been analysed. In another example, it could be that an importing country suspends a processor due to the alleged detection of a banned substance. In this case, the Department of Agriculture and Water Resources may request a traceback due to this detection.

An industry body wishes to conduct a traceback. For example, Australian Pork Limited request a traceback due to information provided by third parties eg. product substitution, provenance claims.

On approval of a traceback request by the Physi-Trace™ management, a sample of the pork and all documentation is sent for analysis. The Physi-Trace™ pork muscle database is used (401). NVD and paper-trial gives indication of date/region of origin (402) and the database is filtered accordingly (403), i.e. only datasets ±3 weeks of the date of the questioned sample are retained. It would be recognized that the revised database now contains data pertinent to sample (404).

The question of tissue type is then considered (405). If the tissue is known, this is entered into the database (406), and the preset elements for the tissue types are recalled (407) and the database is filtered by tissue type (408). It will be appreciated that if the tissue type is pork muscle then the database remains unchanged. If the known tissue type is offal or a processed food then the filtering step (408) is undertaken.

If the tissue is unknown, the steps of entering the data in the database (409), conducting an LDA (410), and filtering (408) are undertaken. A filtered database (B) (411) is the result.

The question whether the sample is processed is then considered (412). If yes, preset elements for processed pork are recalled (413), database (B) is filtered (414) to give database (C) at (415). Database (C) contains only ham or bacon data. If the answer to the above question is no, database (B) is filtered by removing all data for processed samples at step 416. It would be appreciated that database (D) at step 417 now contains either pork muscle data or offal data.

At step 418 the availability of traceability documents and traceability information is then considered.

If such traceability documents and information are available, the database is then filtered accordingly. Steps 419 to 422 deal with the situation where kill sheet information is available. If kill sheet information is available this is entered (419), the database filtered by kill lot (420) to give database (G). An iterative LDA is conducted (422) based on tattoo and a report is generated (423) which may be provided to a user in accordance with the business rules set by the administrator.

Steps 424 to 427 deal with the situation where region information is available. This regional information is entered (424), the database is filtered (425) with filtered Database (F) being the result (426). An iterative LDA is conducted based on the kill lot (427) and this is fed into step 420 which deals with database filtering by kill lot.

Steps 428 to 431 deal with the situation where country information is available. If the information relates to Australia, the steps of entering it in the database (428) and filtering the database (429) are undertaken. Filtered Database (E) is the result (426). An iterative LDA is conducted based on region (431) and this is fed into step 425 which deals with database filtering by region. If the country information does not relate to Australia, a report is generated (432).

If traceability documents and information are unavailable, analysis of the sample is undertaken (433) and an LDA based on the country is conducted (434). If the result indicates the country of origin is Australia then the sample information is added to database (E) for further processing. If the result indicates the country of origin is other than Australia, a report is generated, the traceback concludes and a report is provided to a user in accordance with the business rules set by the administrator.

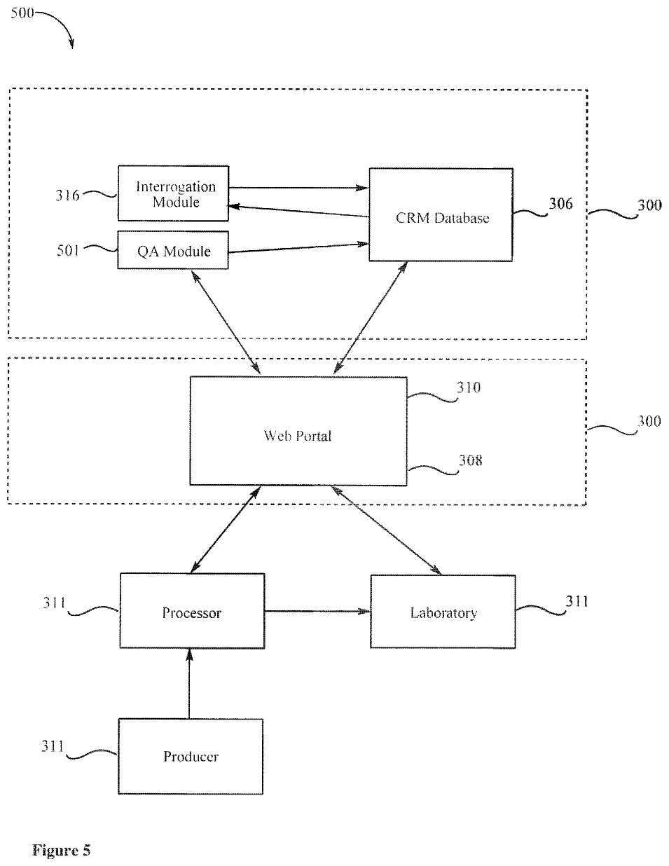

FIG. 5 shows a schematic diagram of one example of a system for reporting the identity of an unknown pig sample, for example an unknown pork muscle sample or an unknown offal sample, as shown in FIG. 4A and FIG. 4B. It will be appreciated that FIG. 5 depicts the relationship between the producer of pig animals, processor of pig animals, and laboratory, which analyses pig samples, as users 311 of the system. Further, the computer system 300, computer system modules (the interrogation module 316 and quality assurance (QA) module (501) are shown in FIG. 5) and the database 306 are depicted. The computer system is implemented by the server 308 having the user interface 315 which is described in FIG. 5 as the Web Portal. The server, itself, may be implemented by any appropriate computing architecture selected from: a standalone PC, client/server architecture, terminal mainframe. A client/server architecture is depicted by the two dashed line boxes in FIG. 5.

It will be appreciated that the database 306, which may be in the form of a customer relationship management (CRM) database, is in communication with the Web Portal through which data/information can be input and output. The pig animal processor may upload details of pig animal samples that have been collected following the sample collection rules described herein to the Web Portal. For example, the pig animal processor may upload details of the tattoo to the Web Portal through an appropriate tattoo field. The pig animal processor may also upload details of pig animal samples that have been collected by scanning of the carcase tag applied by the processor that includes PIC and tattoo information. It will be understood that the system has the functionality such that if there is more than one PIC against a tattoo, or vice versa, then the processor can inform the system which PIC the sample is from by the appropriate data input. The APL PigPass Registration Number will then be identified from the CRM record for that producer, ensuring that the sample is recorded against the correct property. The pig animal processor also provides samples to a laboratory for analysis in accordance with the sample handling and transportation protocols described herein. After the laboratory has processed samples in accordance with the digestion and analytical methods described herein, analytical data, for example in CSV format, may be uploaded to the Web Portal.

As shown in FIG. 5, data may be transmitted to a quality assurance module (501) which processes data as described herein. In this example, the filtered data is transmitted to the database and then to the interrogation module 316 to assess the identity of the pig animal samples. A report providing an assessment of the identity of the unknown pig sample is generated. It will be appreciated that the report can be provided to a user 311, such as the pig animal processor, through the database 306 and the Web Portal with the proviso that the processor has permission to view the report, the permission being set by the administrator. It would be recognised that the computer system may advise the pig animal processor and laboratory of the samples to be analysed through email or similar form of communication.

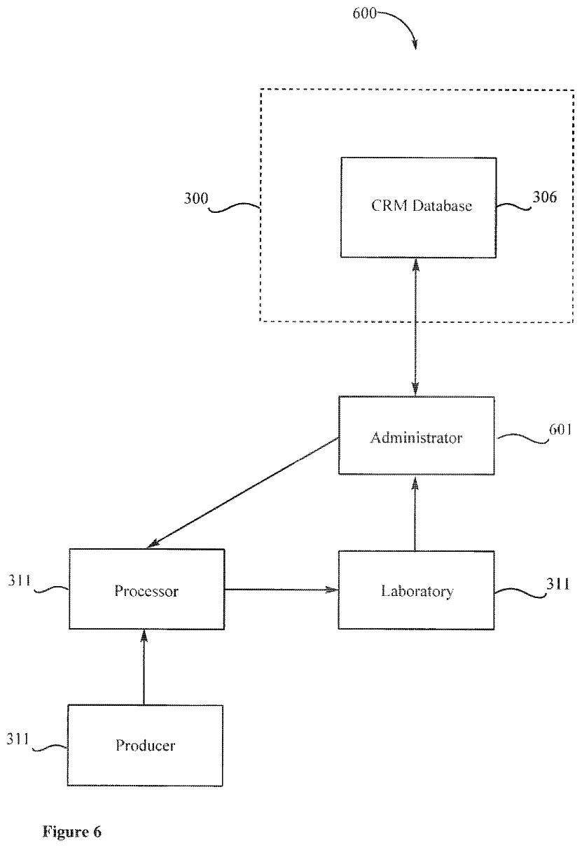

FIG. 6 shows the system of FIG. 5 where quality assurance of data, and interrogation of data to assess the identity of the unknown pig sample is undertaken manually (rather than by the quality assurance and interrogation modules of the computer system). In this example of a system for reporting the identity of an unknown pig sample, data, received by either the processor or laboratory, are input into the database 306 manually by the administrator (shown in FIG. 6 as 601) without the use of the Web Portal. Analytical data is processed for quality assurance manually by the laboratory and interrogation of data is undertaken manually. The report providing an assessment of the identity of an unknown pig sample is generated by the computer system and forwarded to the administrator who then provides the report to a user, in this case the pig animal processor.

### EXAMPLES

### Example 1

Sample Collection, Collection Rules and Sample Transport

The sampling protocol is based on the number of pigs killed in a given week and the number of unique tattoos that appear in that week. For each unique tattoo, ALL pigs killed in that week are totalled. If the total pigs killed for a given tattoo is 1000 or greater in that week, then 10 samples are taken. If the total pigs killed for a given tattoo is between 100 and 999, then 5 samples are taken. If the total pigs killed for a given tattoo is between 30 and 99, then 3 samples are taken. If the total pigs killed for a given tattoo is less than 30, then NO samples need to be taken.

For example, if tattoo RAR appears twice on Monday (with 25 and 25 pigs each lot) and once on Thursday with 51 pigs, then the total would be 101 pigs. Thus 5 samples are required. These 5 samples could be taken on Monday or over Monday and Thursday.

Detailed Protocol

This procedure is relevant for all abattoirs. Note this sampling protocol is a weekly protocol meaning that the number of samples taken is dependent on a cumulative weekly total.

On the Sunday of each week the kill agendas for the whole week (next 7 days) is inspected. All unique tattoos are grouped with the total number of pigs to be killed for that unique tattoo. This is the weekly sampling agenda. For example, consider a week in which the abattoir was operational on 3 days only (see Table 2).

There are 8 unique tattoo from all the kill lots for that week: RAR, GSL, LJS, ABC, XYZ, SGI, SFT and BEC.

Tattoo RAR appears 3 times on the kill sheet of the Jan. 4, 2014 and once on the kill sheet Mar. 4, 2014. The cumulative total of pigs to be killed from RAR in the week will be 170×4=680.

Since 680 pigs falls between 100 and 999, 5 samples need to be taken for tattoo RAR. These 5 samples can be taken whenever the abattoir decides: i.e. ALL 5 samples can be taken from Lot 1 on Jan. 4, 2014, or they could be spread out between the 3 lots on that day or over the 4 lots during the week.

Similarly tattoo GSL appears once on Jan. 4, 2014, 3 times on the Feb. 4, 2014 and once on the Mar. 4, 2014 for a cumulative 1070 pigs to be killed. Thus 10 samples of this tattoo are required. Once again, the samples can be taken from across all 5 kill lots or from just one.

TABLE 2
Three kill sheets representing the forthcoming weeks kill agenda
Arrival Date: Jan. 4, 2014
ARRIVALS

| | | | | |
|---|---|---|---|---|
| 1 | RAR | FARM 1 | 170 | FEMALE |
| 2 | RAR | FARM 1 | 170 | CASTRATE |
| 3 | RAR | FARM 1 | 170 | FEMALE |
| 4 | GSL | FARM 2 | 216 | FEMALE |
| 5 | US | FARM 3 | 136 | MIXED |
| 6 | ABC | FARM 4 | 99 | MIXED |
| 7 | XYZ | FARMS | 10 | MIXED |

Arrival Date: Feb. 4, 2014
ARRIVALS

| | | | | |
|---|---|---|---|---|
| 1 | GSL | FARM 2 | 212 | FEMALE |
| 2 | XYZ | FARM 5 | 15 | MIXED |
| 3 | SGI | FARM 6 | 309 | CASTRATE |
| 4 | GSL | FARM 2 | 210 | CASTRATE |
| 5 | GSL | FARM 2 | 210 | CASTRATE |
| 6 | SFT | FARM 7 | 200 | FEMALE |
| 7 | SGI | FARM 6 | 310 | FEMALE |

Arrival Date: Mar. 4, 2014
ARRIVALS

| | | | | |
|---|---|---|---|---|
| 1 | RAR | FARM 1 | 170 | FEMALE |
| 2 | BEC | FARM 8 | 170 | CASTRATE |
| 3 | BEC | FARM 8 | 170 | CASTRATE |
| 4 | SGI | FARM 6 | 337 | MIXED |
| 5 | GSL | FARM 2 | 212 | CASTRATE |
| 6 | SGI | FARM 6 | 310 | FEMALE |

(1) In this way, determine the number of samples which are required to be taken based on the following plan:
No samples are required for tattoos that contain fewer than 30 pigs over the week. The only tattoo in the kill sheets above in this category is XYZ (25 pigs).
If a tattoo has between 30 and 100 pigs killed within its lot(s) take 3 samples. Note that in the example above, only one unique tattoo (ABC) falls into this category.
If a tattoo has between 100 and 1000 pigs killed within the week, take 5 samples. In the above example, most tattoos fall into this category.
If a tattoo has more than 1000 pigs killed in the week, then take 10 samples.

Note: the samples are to be taken from different carcasses with the tattoo code.

(2) Having determined the number of samples that will be taken in a given week, set aside this number of sample vials for collection. It is recommended that the respective tattoo codes be written with permanent marker on the space provided on the bar code label.

(3) Take the appropriate number of samples from each tattoo code throughout the week. Record the vial bar code (with hand-held bar code reader) and carcass ticket bar code with each sample. Also record the tattoo number on the bar code label to guard against bar code reader failure.

(4) Each sample taken is then added into a spreadsheet, known as the {Abattoir} Physi-Trace™ Samples spreadsheet, (csv or xls spreadsheet) described in the next section.

(5) On the next Sunday, the following week's kill agenda is again inspected and the process repeats.

(6) Samples are to be stored in weekly batches in a freezer (as per “Storage Protocol” section below).

(1) All samples, including samples for storage and samples designated for analysis, are recorded in a csv or xls spreadsheet. This spreadsheet is called the “{Abattoir} Physi-Trace™” Spreadsheet.

(2) Files are named “PT Samples XXX YYMMDD”;
where XXX denotes processor, eg. XXX—Pork Processing Company
YYMMDD denotes final kill date contained within file

(3) Each file contains the following fields:
Kill Date (killdate)
A sample identification Physi-Trace™ Bar Code (physibarcode)
Body Number (bodyno)
Tattoo (tattoo)
Producer Name (name)

An example of the required data in a csv format is detailed below:

killdate,physibarcode,bodyno,tattoo,name

23/11/2010,Pt000011,310,3XXX, Jones' Piggery

23/11/2010,Pt000012,308,3XXX, Jones' Piggery

23/11/2010,Pt000019,293,3XXX, Jones' Piggery

23/11/2010,Pt000031,1825,3XYZ, Smith Pastoral Co.

23/11/2010,Pt000032,1787, 3XYZ, Smith Pastoral Co.

23/11/2010,Pt000033,296, 3XXX, Jones' Piggery

23/11/2010,Pt000034,1776, 3XYZ, Smith Pastoral Co.

Storage Protocol

(1) All samples should be stored at freezer temperatures (−18° C.)

(2) Samples should be stored in monthly groups preferably in boxes for easy identification and access.

(3) Samples are stored for a period of 1 year.

(4) Periodically, APL will request samples for analysis. These samples will be removed and sent to the laboratory nominated by the Physi-Trace™ administrator for processing (see Transportation Protocol below).

(5) Each month, APL will instruct all processors to destroy samples that were collected over one year ago.

(6) The plants are responsible for removal of these samples and their destruction.

Transportation Protocol

Sample Tubes

(1) Boxes of sample collection tubes will be periodically despatched to the abattoirs.

(2) New sample collection tubes will be despatched to the abattoir or if current stockpile of sample collector tubes is running low.

Samples for Analysis

(1) Once samples (for the respective monthly block) have been identified for analysis (this will be initially determined by APL), place sample tubes in a plastic bag and pack the bag in a portable cooler (esky) with dry ice/freezer bricks and seal with tape. If using a polystyrene box then place polystyrene box into a cardboard box to reduce the possibility of damage.

(3) Contact the relevant courier to collect the esky. Samples are to be sent by air courier.

### Example 2

Pork Muscle Analysis and Traceback

An aim of the study presented in Example 2 was to establish if a lower frequency of sampling and analysis could be implemented whilst still maintaining the ability to successfully undertake a traceback investigation for pork muscle back to the processor of origin or tattoo code of origin. This study also aimed to establish the applicability of an existing traceability database to data associated with current samples and thus determine if the existing database could be used as a standalone reference to the elemental profiles of the associated tattoo codes or if regular updating of the database was necessary to maintain its efficacy.

Materials

Laboratory grade nitric acid HNO₃ and 30% v/v hydrogen peroxide H₂O₂ was obtained through Univar from Ajax Finechem Pty. Ltd. Nitric acid was distilled using sub-boiling quartz stills manufactured by Quarzglas Komponenten and Services QCS GmbH, Germany. The final redistilled acids and hydrogen peroxide were analysed as a quality control check, prior to their use, for the concentration of all analytes determined in this study. Sterile polypropylene tubes (50 mL volume) were obtained from Greiner Bio-one and were maintained as single use reaction vessels. Certified Reference Materials (CRM) including National Research Council of Canada (NRC-CNRC) Dogfish Liver CRM (DOLT 4); Fish Protein CRM (DORM 3); and Lobster Hepatopancreas CRM (TORT 2) were used as standards as well NCS DC73347 (China National Analysis Centre) and an in-house pork muscle standard. The in-house pork muscle standard was prepared by taking pork muscle (Weirs Butchers, Nedlands), removing fat and sinew and mincing the muscle in a standard steel ground mincer. The minced muscle tissue was freeze dried and then homogenised using a Braun Mill. Stored pork muscle standard was kept frozen at −9° C.

Sample Preparation and Analysis

Labelled sample collection tubes, containing approximately 10 g of muscle from an individual animal, were received. A sub-sample (approximately 2 g wet mass) of lean muscle was placed into a labelled, pre-weighed reaction vessel. An acid/peroxide solution (6 mL HNO₃ and 2 mL H₂O₂) was added to each vessel and heated at 50° C. overnight. The temperature was increased to approximately 100° C. and the volume reduced to approximately 1 mL. The samples were then made up to 30 mL using high purity 18 MegΩ water.

Reagent blanks, the CRMs DOLT-4 and TORT-2 (National Research Council of Canada), NCS DC73347 (China National Analysis Centre) and an in-house freeze dried pork standard were also included with each batch to ensure accuracy and facilitate data normalisation. Approximately 0.2-0.5 g of each CRM and of the in-house freeze dried standard were accurately weighed into reaction vessels and digested in the same manner as the muscle samples. The elemental concentrations of the solutions were determined as described below in the section Instrumentation.

Instrumentation

The determination of trace element concentrations was conducted using a Thermo Scientific iCAP 6500 Duo Inductively Coupled Plasma Atomic Emission Spectrophotometer and an Agilent 7700x Inductively Coupled Plasma Mass Spectrometer run in standard and/or He collision cell mode. Typical operating parameters are shown below in Table 3 and Table 4.

TABLE 3
Typical operating parameters for ICP-AES

| Parameter | ICP-AES | |
|---|---|---|
| RF Power | 1150 | W |
| Auxiliary Gas Flow | 0.8 | L/min |
| Nebuliser Gas Flow | 0.60 | L/min |
| Coolant Gas Flow | 12 | L/min |
| Flush Pump Rate | 100 | rpm |
| Analysis Pump Rate | 70 | rpm |
| Pump Stabilisation Time | 10 | seconds |
| Peristaltic Pump Tubing | PVC 0.76 | mm diameter |
| 5% v/v HNO₃ Rinse Time | 15 | seconds |
| Sample Flush Time | 25 | seconds |
| Maximum Integration Time | 30 | seconds |

TABLE 4
Typical operating parameters for ICP-MS and ICP-CC-MS

| | ICP-MS | | ICP-CC-MS | |
|---|---|---|---|---|
| RF Power | 1500 | W | 1500 | W |
| Carrier Gas Flow | 1.02 | L/min | 1.02 | L/min |
| Auxiliary Gas Flow | 0.9 | L/min | 0.9 | L/min |
| Nebuliser Pump | 0.13 | rps | 0.13 | rps |
| Interface Cones | Platinum | | Platinum | |
| Omega Bias | −50 | V | −80 | V |
| Omega Lens | 10.5 | V | 8.6 | V |
| Cell Entrance Aperture | −40 | V | −40 | V |
| Cell Exit Aperture | −52 | V | −62 | V |
| Plate Bias | −43 | V | −35 | V |
| Helium Gas Flow | 0 | mL/min | 4.5 | mL/sec |
| Octapole RF | 160 | V | 160 | V |
| Octapole bias | −11 | V | −12.2 | V |
| Quadrupole | −6 | V | −7.2 | V |
| 5% v/v HNO₃ Rinse | 20 | seconds | 20 | seconds |
| Sample Flush Time | 25 | seconds | 25 | seconds |
| Integration Time | 0.1 | seconds | 0.1 | seconds |

Blanks and multi-element calibration standards from Merck Chemicals Australia were analysed at the beginning and end of each analytical run. A wash out solution, consisting of an homogenised sample of remaining sample solutions, was used to tune and condition the ICP-MS prior to analysis, and to washout any standard solution residues at the beginning of the analytical run. In order to account for any instrumental drift (the change in instrument sensitivity as the analytical run progresses) during ICP-AES analysis a drift solution made up of a 5% v/v HNO₃ wash sample and an 18 MΩ deionised water sample was run after every eight samples. This solution facilitates retrospective drift correction in that all samples were linearly corrected for any instrumental drift that has occurred. In order to account for any instrumental drift during ICP-MS analysis, three internal standards were used: beryllium (for analytes ⁷Li to ⁶⁶Zn); rhodium (for analytes ⁶⁹Ga to ¹³⁸Ba); and iridium (for analytes ¹³⁹La to ²³⁸U). In this case, all samples were normalised relative to one another to correct for any instrumental drift that has occurred. To ensure batch to batch comparability, cross over samples from previous analyses were analysed in each analytical run. The quality of the data was assessed using the CRMs (DOLT-4, TORT-2 and NCS DC73347).

A typical sample sequence for an individual analytical run, excluding drift solutions in ICP-AES, is detailed in Table 5.

TABLE 5
Analytical sample sequence with ICP-AES and ICP-MS. Note drift solutions were run
to assess drift in instrumentation detection sensitivity (every eight samples for ICP-AES)

| Sample Type | Purpose |
|---|---|
| Calibration Standards | To convert counts per seconds measured by the |
| | instrumentation to concentrations |
| Wash Out 1-5 | To wash out the tubing and minimise the occurrence of |
| | analytical trains/carry over |
| Blank 1-3 | To determine if any contamination event occurred during |
| | sample preparation and to account for the background |
| | signature |
| Wash Out 6-10 | To wash out the tubing and minimise the occurrence of |
| | analytical trains/carry over |
| 1577c Bovine Liver CRM 1-3 | To determine the accuracy of the digestion and analytical |
| | run |
| Samples 1-n | All samples grouped into tissue type |
| Duplicates 1-n | Three duplicates of each tissue type to determine the |
| | repeatability of measurements |
| Crossovers 1-n | Three duplicates per tissue type and three muscle |
| | crossovers from a previous run analysed to facilitate the |
| | normalisation of data to a given database for comparability |
| | purposes |
| Calibration Standards | To access the accuracy of the calibration and internal |
| | standard normalization |

Data Interpretation

A range of chemometric statistical tools were used to assess the significance of multi-elemental profiles and their relationships to one another. These include linear discriminant analysis (LDA), principle component analysis (PCA), and Wards method of hierarchical clustering. LDAs were conducted using a forward stepwise model with a tolerance level of 0.0001 and a significance level of 5%. To assess the models' accuracy leave-one-out cross validation was performed. Wards method of agglomerative hierarchical was modelled using Euclidean difference based on dissimilarities between categories with automatic truncation. All data interpretation and analysis was conducted using XLSTAT 2012©.

Pork Muscle Traceback

Three batches of samples were provided (Table 6) with kill dates ranging from December 2011 through to August 2012. Data determined for these 706 samples were processed, normalised and incorporated into the existing Physi-Trace™ database.

TABLE 6
Sampling dates for samples provided for analysis
(sample numbers detailed in parentheses)

| | Plant A | Plant B |
|---|---|---|
| Batch 1 | May 2012 (69) | May 2012 (184) |
| Batch 2 | February 2012 (69) | December 2011 (123) |
| Batch 3 | August 2012 (66) | March 2012 (195) |

Eight samples were selected from each batch to be treated as unknowns, using samples from the same tattoo codes for all three batches. These tattoo codes were chosen to represent both processors, using both high frequency (Farm A and Farm B from Plant A; Farm X and Farm Y from Plant B) and low frequency tattoo codes (Farm C and Plant D from Plant A; Farm U and Farm W from Plant B).

Tracebacks were undertaken for each “unknown” sample for each batch, relating the samples back to four separate database groups using the protocols described herein. These database groups included the data within a single batch (a reduced, current database), the three new batches (an extended, current database), the existing Physi-Trace™ database without the inclusion of new data and finally to the entire database (the existing database plus that current batch).

Identification of Processor of Origin

All unknown samples for the three batches were successfully related back to their processor of origin using forward stepwise LDA with reference to three of the four database groups. For all database groups, the discriminant model used to identify the processor of origin exhibited higher correct classification rates (99% compared to 93%) when data from new batches were considered, as opposed to any combination involving the existing Physi-Trace™ database. Although the variation observed in the classification rates is small, it was attributed to the reduced number of groups (two processors instead of four) contained in the new batches and thus a reduced spread of data within each group (lower sample numbers) such that the internal variation that occurs over time is not taken into consideration. Furthermore, the two processors involved (Plant A & Plant B) have considerably different elemental due to large geographical differences due to their locations. This was particularly evident when undertaking processor classification on the data without inclusion of any new data batches (only relating the unknowns to the existing Physi-Trace™ database), resulting in misclassification for 50% of the unknown samples from Plant B. Consequently, the broad classification of an unknown sample to its processor of origin appears possible when the processor exists within the database, so long as some current data are available. However, the combination of current samples with an existing current database results in the most robust statistical model.

Identification of Tattoo Code of Origin

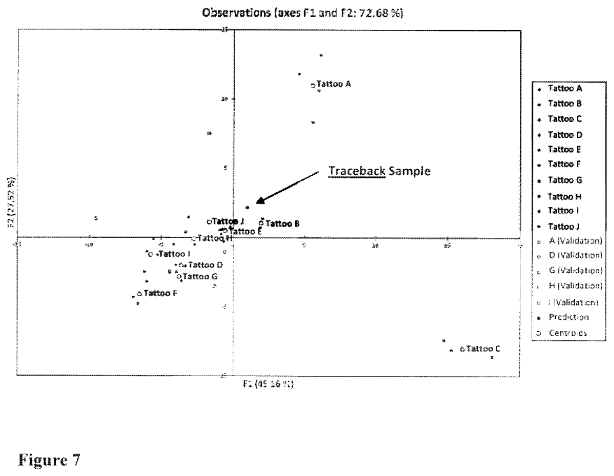

When the classification of an unknown sample to its tattoo code of origin was investigated using Plant B data from December 2011, it was possible to generate a discriminant model that correctly classified 96.7% and 100% of the estimation and validation sets respectively, correctly predicting that this particular unknown sample originated from tattoo code Farm W. The discriminant plot associated with this model is included as FIG. 7.

For other unknowns, the small number of samples available to construct the discriminant model resulted in undefined, over-fitting models. It would be appreciated that for a successful traceback of pork muscle to tattoo code of origin, it is important that sufficient data is included in the database to appropriately define the variation in each tattoo group population. Thus, to improve the ability to classify an unknown sample, additional samples should be included in the initial construction of the discriminant model. Preferably, there cannot be a significant variation between the profiles of each of the groups (tattoo codes) for these additional samples as compared to the profiles for the current samples.

Testing for the Requirement of Regular Updating of Database

As noted above, this study also aimed to establish the applicability of an existing traceability database to data associated with current samples and thus determine if the existing database could be used as a standalone reference to the elemental profiles of the associated tattoo codes or if regular updating of the database was necessary to maintain its efficacy. A similar series of traceback investigations were undertaken using the same eight samples from each batch as was used previously. The classification of these samples was interrogated with reference to all three new batches of data (706 samples), an existing database (2982 samples) and a combination of their associated new batch of data as well as the existing database.

When the reference database for the traceback investigations was expanded to include all data collected during the National Livestock Traceability Performance Standards Project (3 batches, 706 samples), an improvement in the success rate was observed. Of the 24 traceback investigations, four unknowns were successfully classified to their processor of origin. These four unknowns were associated with both processors (one from Plant B and three from Plant A) and included high and low frequency tattoo codes. This improved success for the tracebacks can be attributed to the additional data facilitating greater definition of the variation within and between tattoo codes. However, the 17% (4/24) success rate for traceback investigations does not match the success achieved in previous studies which utilised the existing Physi-Trace™ database. Consequently, the traceback investigations were repeated, with comparison only to the existing Physi-Trace™ database. This resulted in correct classification of two out of 24 (8%) unknown samples to their tattoo code of origin. Therefore, despite the improved definition of the variation between and within tattoo codes, it appears that the existing Physi-Trace™ database does not sufficiently represent the current data. Thus a combination of the existing Physi-Trace™ database with new data from the National Livestock Traceability Performance Standards Project was investigated.

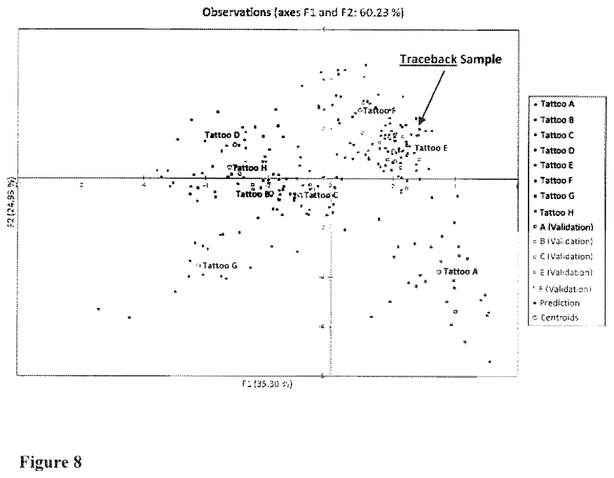

When Plant B samples with December 2011 kill dates were considered, the majority of tracebacks were successful. Three of the four unknown samples could be correctly classified to their tattoo code of origin, often generating discriminant models with high (>90%) correct classification rates for estimation and validation sets with similarly high prediction probabilities. For example, when the traceback was undertaken on a sample from tattoo code Farm X, a discriminant model was constructed using 21 analytes that correctly classified 94.5% and 93.3% of the estimation and validation sets respectively. Using this model, the unknown sample was classified to Farm X with 98.3% probability. The discriminant plot associated with this model is detailed in FIG. 8. With regards to the one unknown sample not correctly classified to its tattoo code of origin, the sample appeared as an outlier rather than misclassifying to another tattoo code. Furthermore, when other samples from that tattoo code were examined, correct classification was possible. This result supports the current analytical protocol for traceback samples whereby a minimum of three replicates are analysed to ensure outliers do not inhibit correct classification of unknown samples.

With regards to results obtained from other batches of data with kill dates ranging from February 2012 to August 2012 (three to nine month discrepancy from the current database) less optimal results were obtained. Of the remaining 20 tracebacks across the batches, only four (20%) resulted in correct classification of the unknown to their tattoo code of origin. These four tracebacks, evenly distributed between the batches, featured low correct classification rates (50-80%) for estimation and validation sets and poor prediction probabilities (40-70%) with regards to the origin of the unknown. Interpretation of these results indicates that there is a relationship between the currency of a database and likely success of a traceback. When a current database is available (as observed with the Plant B batch with kill dates of December 2011) a traceback investigation can be completed successfully.

The person skilled in the art would recognise that minor variations in the elemental profiles of the tattoo codes will occur over time and can be attributed to a number of factors including potential changes to feed and water sources and environmental events. As a consequence of this, it is essential to relate any unknown sample to a current database of sufficient size and quality (approximately 6-12 months of data) to maximise the ability for a traceback investigation to be conducted successfully. In conclusion, it will be appreciated that regular sampling and analysis of reference samples allows for accurate identification of pig samples.

### Example 3

Traceback of Offal

An aim of the studies presented in Example 3 was to investigate offal sample traceability for region of origin; state of origin or farm of origin assessment and assignment of unknowns. A further aim was to determine whether a muscle-specific database could be used for offal traceability and assignment of unknown offal samples.

Materials

Laboratory grade HNO₃, HClO₄, HCl and 30% v/v H₂O₂ were obtained through Univar from Ajax Finechem Pty. Ltd. Nitric, perchloric and hydrochloric acids were all distilled using sub-boiling quartz stills manufactured by Quarzglas Komponenten and Services QCS GmbH, Germany. The final redistilled acids and hydrogen peroxide were analysed as a quality control check, prior to their use, for the concentration of all analytes determined in this study. Sterile polypropylene tubes (50 mL volume) were obtained from Greiner Bio-one and were maintained as single use reaction vessels. National Institute of Standards and Technology (NIST) Bovine Liver 1577c CRM was used to ensure accuracy of analytical data and an in house standard of homogenized freeze dried pork muscle (prepared as described in Example 2) was also used to check batch to batch variations of all analytes not certified in the NIST standard. Multi-element standards were provided by Merck Chemicals Australia.

Sample Preparation

Approximately 2 g (wet weight) of each sample, 0.5 g Bovine Liver 1577c Certified Reference Material (CRM) and 0.5 g of an internal muscle in-house standard were accurately weighted to four decimal places and cold digested for 48 hours in 6 mL of HNO₃. After the initial oxidation had taken place 2 mL of H₂O₂ were added and the samples were transferred to a water bath to reflux for 24 hours at 90° C. Following this, an additional 2 mL of H₂O₂ were added and the samples were left to reflux for a further 2 hours. Solutions were then brought down to approximately 2 mL final volume before being made up to 30 mL using 18 MegΩ deionised water. Once dissolved all samples were appropriately diluted in 2% quartz still redistilled HNO₃ containing 2 μgmL⁻¹ of Rh and Ir as internal standards to monitor analytical drift.

Three blanks, three CRM's and three in-house standards were treated in the same manner for each analytical run. An analytical run consisted of approximately 180 samples. Fifteen percent of samples were analysed in duplicate and a minimum of three cross-over samples from previous runs per tissue type were incorporated into each batch. The replicate samples and cross-over samples were analysed at the end of each analytical run to assist in determining true reproducibility of analytical data and batch variation between analytical runs.

Analysis

Solutions were chemically analysed as described in Example 2. Multi-element standards from Merck Chemicals Australia and sample dissolution blanks were analysed at the beginning and end of each analytical run allowing for the calibration of analytical data. Prior to each sample a 20 second rinse of 5% v/v HNO₃ was conducted followed by a 25 second rinse with the sample before data acquisition commenced. To assess analytical drift during ICP-AES analysis, a drift solution was analysed every 12 samples followed by a 5% v/v HNO₃ and 18 MegΩ deionised water rinse. In total a suite of 62 elements were determined.

Data interpretation was carried out as for Example 2 with the added techniques. Mathematical integration of offal multi-elemental profiles to their muscle-specific equivalence was investigated using linear regression modelling and then conversion ratios. For each analyte of interest a median multiplication factor was determined for a data set of 127 pigs. These factors enabled the conversion of offal-specific concentrations to their muscle-equivalent concentration. The error of these factors was determined by the relative standard deviation observed in the factor across all swine investigated.

Chemical Traceability of Offal

Assignment of swine tissue was assessed for a multitude of scales from region of origin to farm of origin. The accuracy of assignment is presented as a percentage of correctly cross validated samples in linear discriminant analysis (Table 7). All edible pork offal types had a similar accuracy of assignment to geographic origin of rearing as their respective muscle tissues. The major key discriminatory elements for provenancing of the swine tissues were rubidium, strontium, caesium, selenium, arsenic, potassium, thallium and cobalt which show a strong relation to geology of a site, climate and anthropogenic influence.

TABLE 7
Percentage correct cross validation of swine tissue samples to
geographic region, state and farm of origin using LDA modelling.

| | MUSCLE | TONGUE | STOMACH | HEART | LIVER | KIDNEY |
|---|---|---|---|---|---|---|
| Region of Origin | 100 | 100 | 99.19 | 100 | 100 | 100 |
| W AUS | 100 | 100 | 100 | 100 | 100 | 100 |
| NE AUS | 100 | 100 | 96.15 | 100 | 100 | 100 |
| SE AUS | 100 | 100 | 100 | 100 | 100 | 100 |
| State of Origin | 88.2 | 83.3 | 83.7 | 86.61 | 91.3 | 89 |
| WA | 100 | 100 | 100 | 100 | 100 | 100 |
| QLD | 100 | 95.8 | 96.2 | 100 | 100 | 100 |
| NSW | 87.3 | 89.3 | 75.9 | 84.9 | 90.9 | 84.9 |
| VIC | 55 | 45 | 70 | 60 | 88.4 | 80 |
| SA | 90 | 75 | 70 | 80 | 90 | 80 |
| Farm of Origin | 89.9 | 84.88 | 78.66 | 83.88 | 90.72 | 91.26 |
| WA | 100 | 96.4 | 89.3 | 96.4 | 78.6 | 89.3 |
| QLD | 100 | 100 | 92.5 | 100 | 100 | 100 |
| NSW | 89.5 | 88 | 81.5 | 88 | 100 | 92 |
| VIC | 80 | 70 | 65 | 80 | 75 | 85 |
| SA | 80 | 70 | 65 | 55 | 100 | 90 |

Region of Origin Assessment

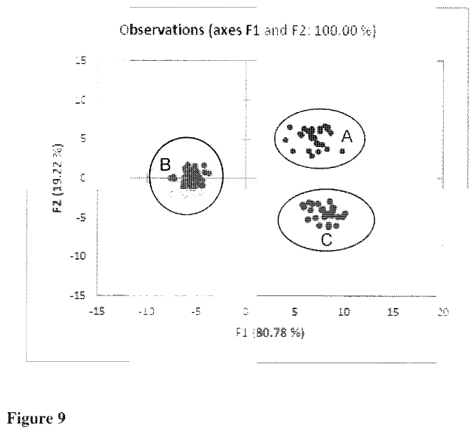

Linear discriminant analysis of elemental data for swine from different regions of origin produced a cross validation of greater than 99% for all tissue types studied (see Table 7). A representative linear discriminant analysis can be seen in FIG. 9 demonstrating the clear grouping of samples into regions of origin. Not only do vast spatial differences exist between Western Australia, North Eastern Australia and South Eastern Australia, the bioregions within these areas have unique climatic, lithogenic and biotic influences.

State of Origin Assessment

Assignment of swine tissues to a given state of origin exceeded a correct percentage cross validation of 83% for all tissue types investigated (Table 7). The ability to clearly distinguish swine based on the chemical profile of a given tissue to a state of origin was state specific with Queensland and Western Australia demonstrating a cross validation exceeding 95% for all tissue types.

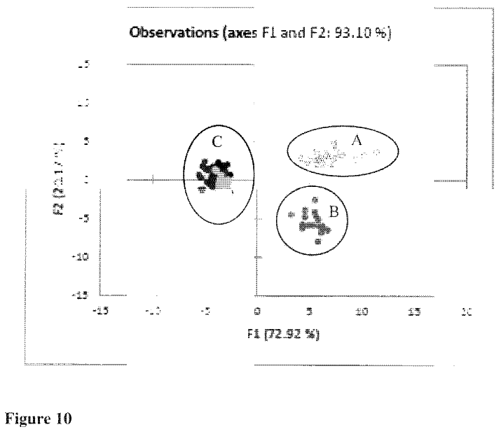

The unambiguous separation of Queensland and Western Australian swine tissues based on their chemical composition was observed in the Region of Origin assessment detailed above with reference to FIG. 9. These two states of origin are the only representatives in their region (North East Australia and Western Australia). In contrast, data for the South Eastern Australian region is a composite of data for tissues from swine growing in three different states (Victoria, New South Wales and South Australia). Consequently when data for all five states are investigated using LDA, considerable overlap in the distribution of Victoria, New South Wales and South Australia in the model was observed (FIG. 10).

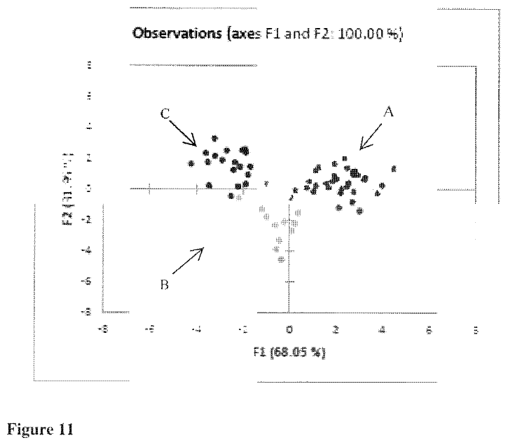

Correct cross validation of tissues to state of origin was higher for the New South Wales and South Australian samples exceeding 75% and 70% respectively for all tissue types investigated, but lower for Victoria. By employing further iterative steps assignment of swine tissues to South Eastern Australian States of Origin was improved (FIG. 11). Overall correct assignment of tissues to a South Eastern Australian state of origin exceeded 73% for all tissue types and cross validation for New South Wales and South Australia samples were increased to greater than 84% and 80% respectively (see Table 7), but lower for Victoria.

The skilled addressee would understand that state borders are arbitrarily assigned political lines that do not reflect a natural change in biome or environmental conditions that would aid in discriminating samples. For the farms located in South Eastern Australia, they were located primarily in the same bioregion and, as such it would be conceivable that the chemical composition would be similar between swine from these three states. The results support this theory. With the exception of rubidium no key geographic geological markers were used. This suggests a similar geological profile between the states. Furthermore, the two major processors in the area are supplied by producers located in more than one state. This spread of farms across the states not only means movement of swine is more common, it also suggests farming practices and thus feeding regimens will be similar for farms from the same producers. If little natural geographic variation exists between the three states of origin, and farming practices are either too similar between states, or too similar between farms within a given state, classification of swine product to a state of origin is not as unambiguous in the case of Queensland and Western Australia.

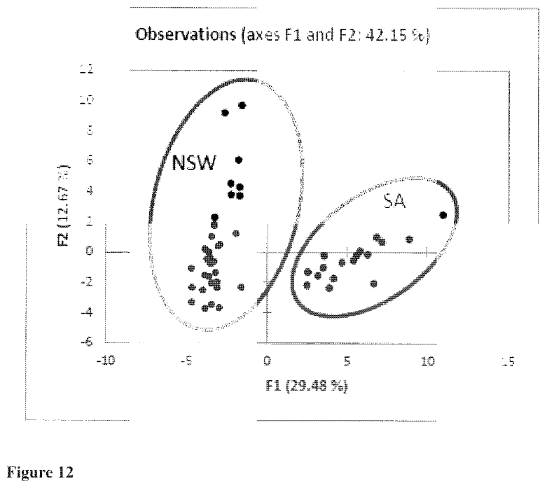

Also, sampling procedures must be taken into account. Sampling of all Victorian animals occurred interstate post transportation to either a plant in South Australia or a plant in New South Wales. Any integration of the chemical signature from the state of slaughter during this transport period would result in an intermediate signature for Victoria swine that is attributable partly to their state of rearing (Victoria) and partly due to their state of slaughter (New South Wales or South Australia). This integration would result in an overlap in the data fields for tissues (as expressed in the LDA plots) from Victoria with both South Australia and New South Wales. This theory is supported by the fact that all tissues collected from South Australian and New South Wales swine are clearly distinguishable from one another based on their chemical profile (FIG. 12). To elucidate a clearer distinction between tissues collected from Victorian farms of origin and those of the surrounding states, additional sampling of Victorian farms is necessary.

Farm of Origin Assessment

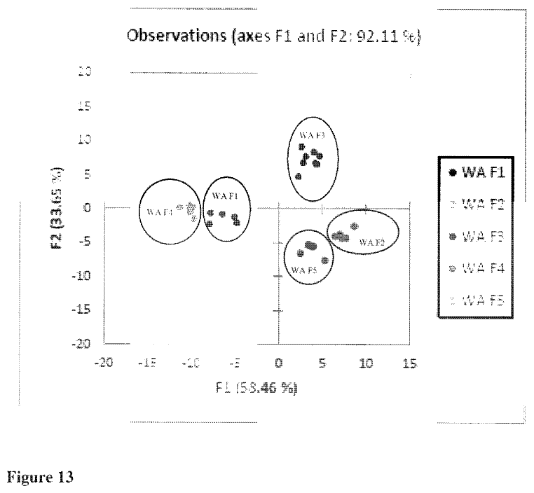

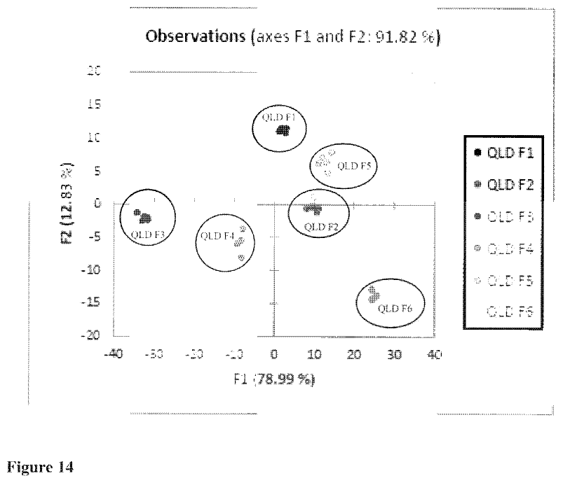

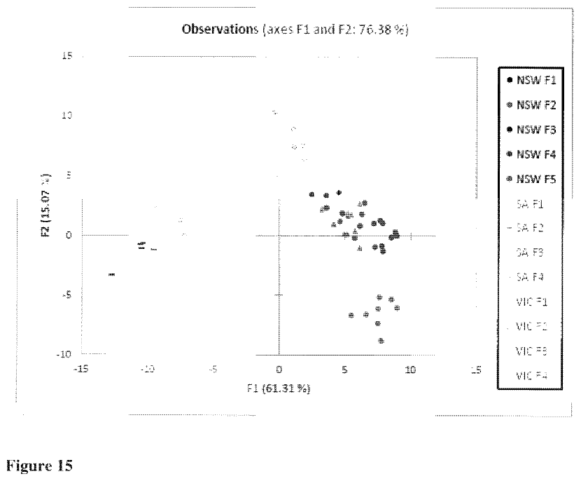

An iterative approach was developed to enable samples to first be classified to region or origin, and subsequently to farm of origin using a reduced dataset. This iterative approach proved more successful than assigning animals to farm of origin using the entire database. Correct cross validation of samples using the iterative approach reached 100% for various Western Australian and Queensland tissue types (Table 7). Representative LDA's for Western Australian and Queensland swine are presented in FIG. 13 and FIG. 14 respectfully. They clearly demonstrate the separation of farms was achievable based on the multi-elemental profile of the tissues. Cross validation was still lower in the South Eastern states of origin due to the larger number of samples and groups in the analysis cohort. The increase number of groups resulted in overlap of the multi-element profile for the different farms (FIG. 15). However, two distinct groupings of farms were identified in the linear discriminant analysis of South Eastern Australian farms. The first group consisted of all South Australian farms and the two Victorian farms that were sampled at the South Australian abattoir. The second group consisted of all tissues from New South Wales farms and the two Victorian farms sampled at the South Australian abattoir. This grouping further supports the need to sample additional Victorian swine to determine the possibility of defining Victorian animals based on their multi-elemental profile.

Assignment of Unknowns

Leave one out cross validation of the models presented above demonstrated the potential to use LDA's of multi-elemental data to distinguish swine tissue samples to region, state and farm of origin. In this study, an investigation of the ability to predict the geographic origin of edible unknown swine offal samples was undertaken. Two swine from each state were randomly selected from the data set and removed to be analysed as unknowns using the region of origin iterative process described previously. The predicted farm of origin for each sample is detailed in Table 8 together with the known identity for the sample. All samples were correctly assigned to region of origin. Furthermore all Queensland, Western Australian and New South Wales samples were also correctly assigned to state of origin. Farm of origin assignment was high for these three states. There were some misclassifications which are attributed to similar chemical profiles between the assigned farm and the known farm of origin. It would be appreciated that with further sampling enabling a greater incorporation of a farm's true chemical signature, multi-element traceability to a higher level of classification can be achieved. The results show that with the database collected during this work, the potential to discriminate farm of origin for New South Wales, Western Australia and Queensland swine tissue with a low error of misclassification has been realistically demonstrated.

TABLE 8
Predicted farm of origin from ten unknown swine muscle, tongue, stomach, heart,
liver and kidney tissue samples from Queensland (QLD), Western Australian (WA),
New South Wales (NSW), Victorian (VIC) and South Australian (SA) states of
origin. Iterative analysis using a region of origin prior classification was conducted.

| | Muscle | Tongue | Stomach | Heart | Liver | Kidney | Identity |
|---|---|---|---|---|---|---|---|
| Sample 1 | QLD F2 | QLD F5 | QLD F2 | QLD F2 | QLD F2 | QLD F2 | QLD F2 |
| Sample 2 | QLD F5 | QLD F5 | QLD F5 | QLD F5 | QLD F5 | QLD F5 | QLD F5 |
| Sample 3 | WA F3 | WA F3 | WA F3 | WA F3 | WA F3 | WA F3 | WA F3 |
| Sample 4 | WA F5 | WA F5 | WA F5 | WA F5 | WA F3 | WA F5 | WA F5 |
| Sample 5 | NSW F1 | NSW F1 | NSW F1 | NSW F1 | NSW F1 | NSW F1 | NSW F1 |
| Sample 6 | NSW F2 | NSW F2 | NSW F2 | NSW F2 | NSW F2 | NSW F2 | NSW F2 |
| Sample 7 | NSW F4 | NSW F4 | VIC F2 | VIC F2 | NSW F4 | NSW F4 | VIC F1 |
| Sample 8 | VIC F4 | SA F3 | VIC F3 | SA F1 | VIC F4 | SA F1 | VIC F4 |
| Sample 9 | VIC F4 | SA F4 | SA F2 | SA F2 | SA F3 | VIC F3 | SA F2 |
| Sample 10 | SA F4 | SA F4 | SA F3 | SA F3 | SA F4 | SA F2 | SA F4 |

Influence of Sex Based on Multi-Elemental Profiles

Females Versus Intact Males

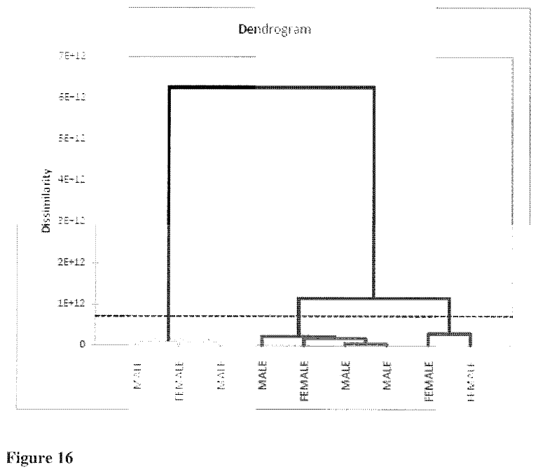

Agglomerative hierarchical clustering for muscle, tongue, heart, liver and kidney tissue identified no statistical difference between the multi-elemental chemical signature of female and intact male swine from the same farm. A representation of these relationships is presented in FIG. 16. Statistical analysis of individual elements supported this finding for muscle with no elements showing a significant difference between the concentrations in the intact male versus the female swine. However, despite the inability to separate males from females using multivariate analysis for tongue, heart, liver and kidney, several elements were identified to be significantly different between male and female swine (Table 9). For tongue samples, the concentration in intact male swine was higher than that in the female swine for all significantly different elements. In contrast, the concentration of all significantly different elements in heart, liver and kidney, were lower in the male than in the female swine.

TABLE 9
Elements displaying significant differences between male and female
tongue, stomach, heart, liver and kidney samples. Level of significance
between the sexes is indicated as a P value.

| Tongue | Stomach | Heart | Liver | Kidney |
|---|---|---|---|---|
| Mo (0.045) | Ag (0.003) | As (0.001) | As (0.002) | As (0.019) |
| V (0.034) | B (0.002) | Cd ((0.047) | Li (0.031) | K (0.037) |
| | Bi (0.014) | Ru (0.045) | Mo (0.016) | Li (0.010) |
| | Ce (0.015) | Tb (0.0003) | | P (0.048) |
| | K (0.039) | | | S ((0.015) |
| | Lu (0.043) | | | Se (0.026) |
| | Ni (0.012) | | | |
| | Pb (0.007) | | | |
| | Zr (0.034) | | | |

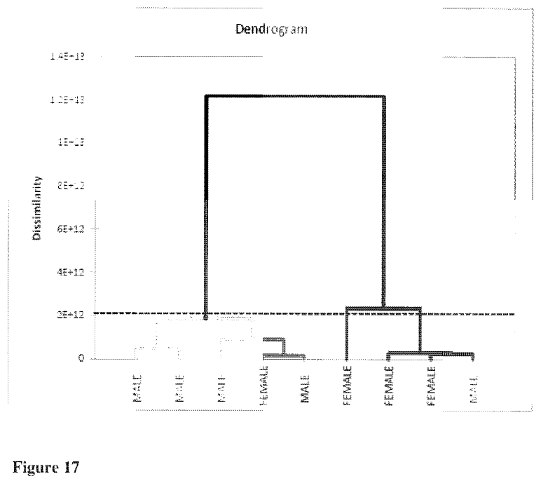

Agglomerative hierarchical clustering for stomach tissue identified two major categories of animals, separating the intact male swine from female swine for all except two animals (FIG. 17). This clustering resulted from significantly higher concentrations of silver, boron, bismuth, cerium, lutetium, nickel, lead and zirconium and significantly lower concentrations of potassium found in intact male swine compared to females. It would be appreciated that by increasing the sample size would improve classification statistics.

Females Versus Immunocastrated Males

Significant differences were observed between the immunocastrated male and female signatures for specific elements (Table 10). For the heart, concentrations of lithium were significantly higher and yttrium was significantly lower in immunocastrated males than in female swine. For liver samples concentrations of lithium, tungsten and uranium were higher in immunocastrated males than females, while concentrations for boron, sulphur and calcium were lower. For lithium in the liver tissue, a significant difference was also recorded between the concentrations of intact male and female swine. However, in both instances the concentrations in the tissues were lower in the intact males than the females. This is in contrast to immunocastrated males and females where the concentration was higher in the immunocastrated males than the females.

TABLE 10
Elements displaying significant differences between immunocastrated
male and female muscle, heart, liver and kidney samples Level of
significance between the sexes is indicated as a P value

| Muscle | Heart | Liver | Kidney |
|---|---|---|---|
| K (0.019) | Li (0.001) | B (0.041) | As (0.043) |
| Mg (0.001) | Y (0.041) | Ca (0.031) | Bi (0.010) |
| P (0.001) | | Li (0.003) | Co (0.023) |
| S (0.013) | | Cu (0.022) | Cs (0.048) |
| Se 0.03) | | S (0.044) | U (0.001) |
| Ti (0.038) | | W (0.034) | Hg (>0.001) |
| Tl (0.038) | | | Li (0.005) |
| | | | Ni (0.011) |
| | | | U 0.035) |
| | | | V (0.007) |
| | | | Z 0.045) |
| | | | Zr (0.002) |

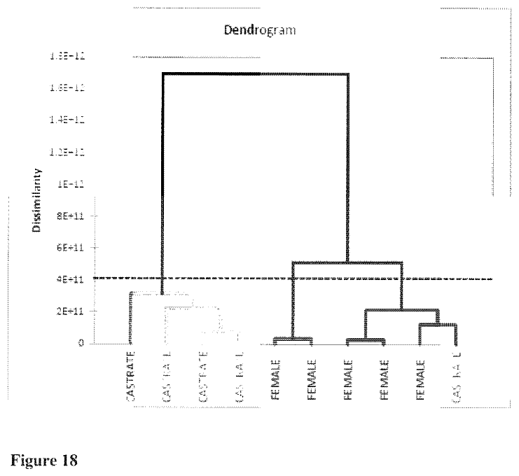

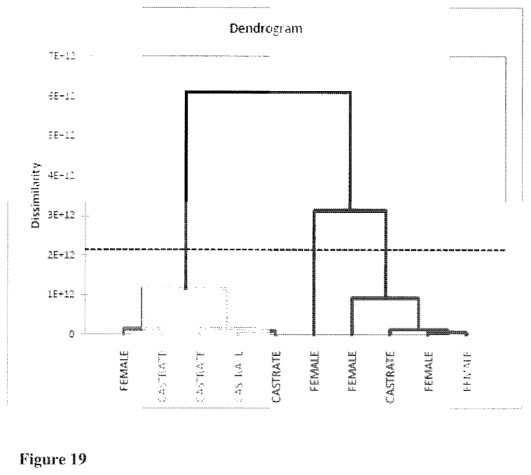

Agglomerative hierarchical clustering resulted in the separation of immunocastrated male and female swine for both muscle (FIG. 18) and kidney (FIG. 19) tissue samples. The clustering of samples based on sex for muscle was a result of significantly lower concentrations of magnesium, phosphorus, sulphur, potassium, titanium, selenium and thallium in immunocastrated males compared to females. These findings were not consistent with intact male versus female data presented earlier where no significant differences in elemental concentrations were identified between intact males and females for muscle tissue. Clustering of individuals into immunocastrated males and females for the kidney tissue resulted from significantly higher concentrations of lithium, vanadium, cobalt, nickel, copper, zinc, zirconium, caesium, terbium, mercury, bismuth and uranium in immunocastrated males than females and significantly lower concentrations in arsenic in immunocastrated males versus females (Table 10).

In general, the muscular tissues of muscle, tongue and stomach had higher concentrations of elements in the intact male than the female swine but had a lower concentration in immunocastrated males than females where a significant difference was identified. Conversely heart, liver and kidney tissue had lower concentrations of elements in the intact male than the female swine, but higher concentrations in the immunocastrated swine than the female swine where a significant difference was observed. These variations in the chemical composition between intact males, immunocastrated males and females were attributable to both differential lean muscle and fat development and the potential differences in thyroid function of the animals altering their metabolism.

Geographic Assignment of Swine to Farm of Origin

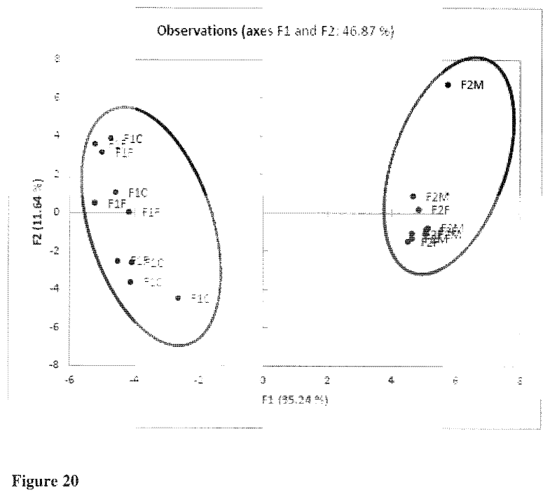

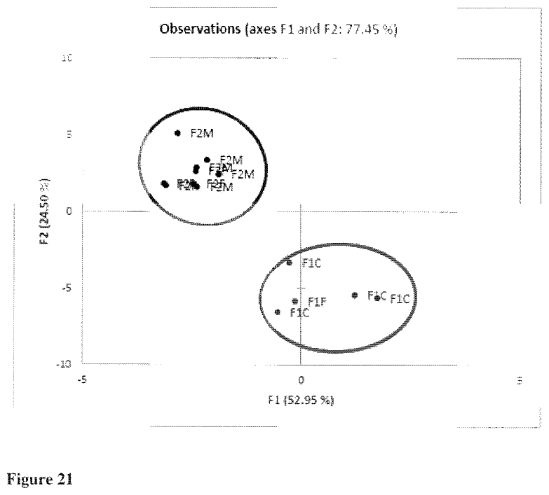

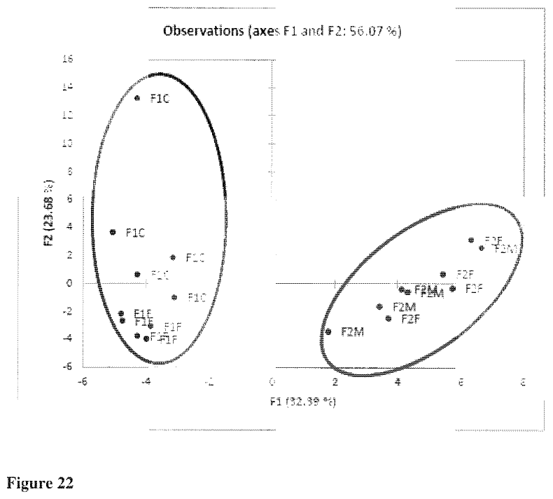

For those tissue types where a multivariate analysis found a significant difference in the chemical composition between the different sexes of swine, a further investigation into the extent of intra-farm variation as a result of sex was investigated in relation the extent of inter-farm variability of swine tissues observed between two different farms of origin. When principle component analysis was performed on muscle (FIG. 20), stomach (FIG. 21) and kidney (FIG. 22) samples from swine originating from the two different farms of origin, the variation in multi-element signatures within the farm caused by sexual difference in trace element accumulation was smaller than the between farm variation in the chemical signatures observed in swine from the different farms. The findings demonstrate that differences in the accumulation of trace elements between intact males and females, or between immunocastrated males and females does exist. To investigate the impact of sex on the chemical signature of swine more fully, it would be appreciated that a more detailed study; one in which intact males, females, surgically castrated males and immunocastrated males can be sampled from a single source with limited variation in nutrition throughout the study is needed.

Use of a Muscle-Specific Database for Offal Traceability

Intra-Specific Trace Element Variation

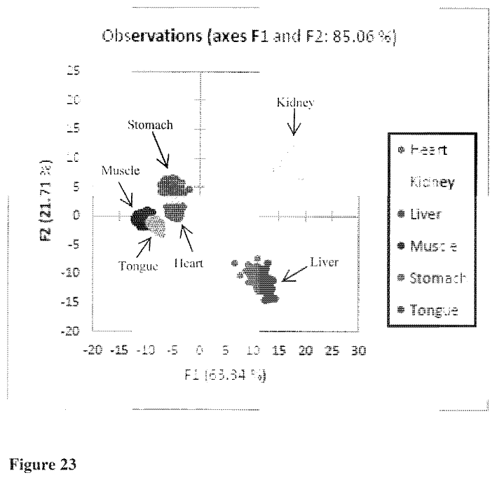

Chemical composition was variable between the different tissue types investigated (FIG. 23). Linear discriminant analysis identified that the analytes contributing to the major differences between tissue types were the essential elements including sodium, manganese, magnesium, phosphorus, sulphur, potassium, molybdenum, calcium, selenium, zinc and iron. This variation was attributable to accumulation of selected elements to significantly higher concentrations in stomach, heart, liver and kidney tissues than that in muscle. The variable accumulation of elements between different tissue stores was associated with the differential physiological and metabolic properties of the investigated tissue types. Due to such differences, direct integration of the offal specific multi-elemental profiles into a muscle specific database would hinder accurate trace back for edible swine offal products but this can be circumvented by using data normalisation steps.

Standardisation of Trace Element Profiles

Because of the different concentrations of trace elements present in the various tissue types, standardization of multi-elemental profiles was required to facilitate the use of a single chemical database for all swine tissues. Mathematical integration of offal multi-elemental profiles to their muscle-specific equivalence was investigated using linear regression modelling and then conversion ratios. Conversion ratios were ultimately determined to be the most appropriate modelling technique.

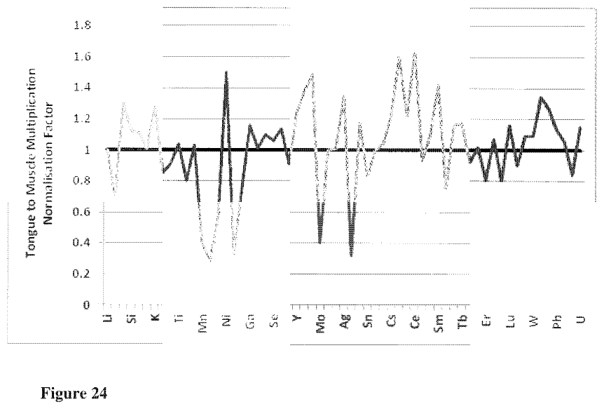

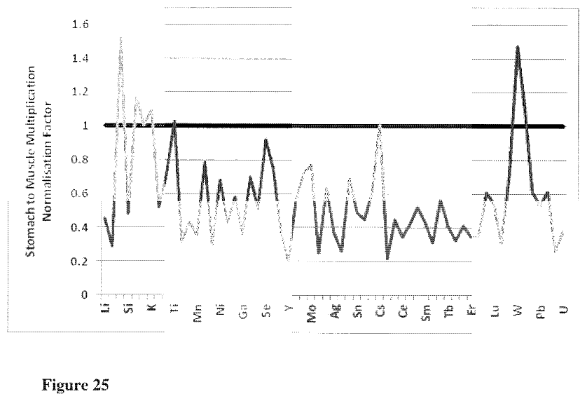

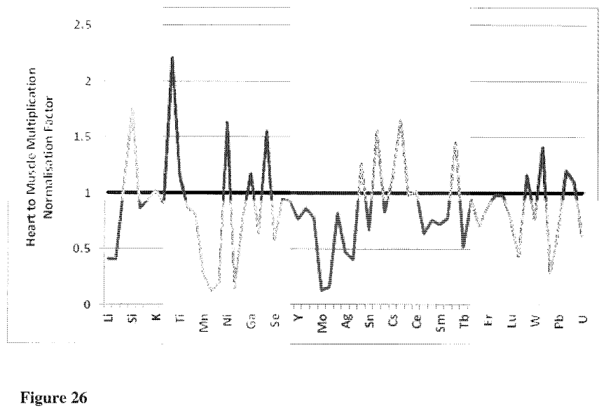

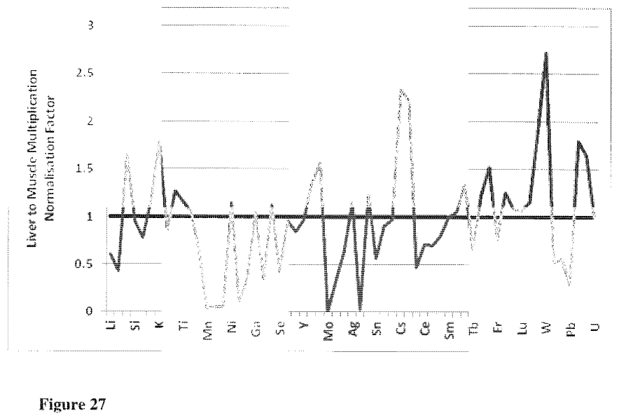

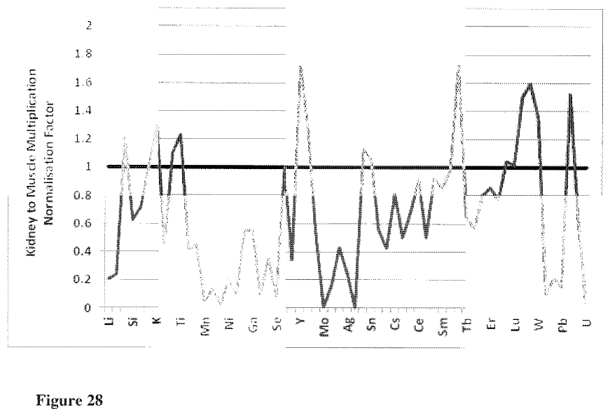

An approach using conversion ratios was conducted by calculating a multiplication factor that enabled the normalisation of the chemical concentration in one tissue type, back to the theoretical muscle profile for the individual animal. The factor profile for tongue, stomach, heart, liver and kidney is detailed in FIGS. 24 to 28 respectively. For all values less than one, the chemical composition in the tissue type prior to normalisation was higher than in the muscle, and for all factors greater than one, the concentration in the tissue type prior to normalisation is lower than in the muscle tissue. The smallest amount of correction required to normalise data to the muscle equivalent concentration was required for tongue (FIG. 24). The largest amount of correction to normalise values to the muscle equivalent was required for kidney tissue (FIG. 28), with a normalisation factor as low as 0.0035 for Cd in the kidney when compared to 0.32 for cadmium in the tongue (a normalisation factor of unity would indicate that the tissue type is equivalent to muscle with respect to a particular trace element).

Traceability of Swine Offal Using a Muscle Specific Database

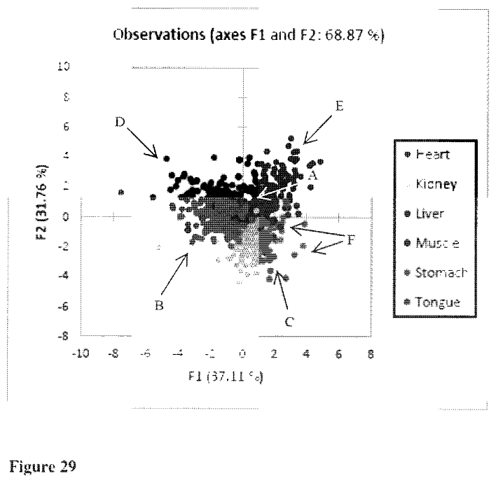

Normalisation of offal tissue to their muscle-equivalent concentrations using the multiplication factors described above brought the multi-elemental profiles for edible offal into line with their muscle equivalent. This is evidenced by an overlap of samples from different tissue types when analysed using linear discriminant analysis (FIG. 29). Not only were a large number (49) of analytes needed to create the model, the highest partial R² value determined was only 0.20 for Th. This demonstrates a low ability for the model to distinguish tissue types based on any of the variables provided thus indicating low variability in the element concentrations between the six different swine tissue types post normalisation.

To assess the robustness of the normalisation factors developed, edible offal samples from a single Western Australian farm of origin were selected as unknowns. A Western Australia farm of origin was used as the model required that, using data for individual organ types, a region could be clearly distinguished from all other regions of Australia and that farms within that area could also be separated based on the tissues' chemical signatures. By selecting a farm that shows clear assignment to region and farm of origin ensured that the robustness of the normalisation factors, and not the concept of multi-element traceability, were tested. The principal of the concept was that if farms overlapped then no amount of normalization would separate them but if farms remained separate after normalization this would at least show that the normalization concept could work. The results for the prediction of each offal type to region and farm of origin before and after normalization to their muscle equivalent concentrations is presented in Table 11.

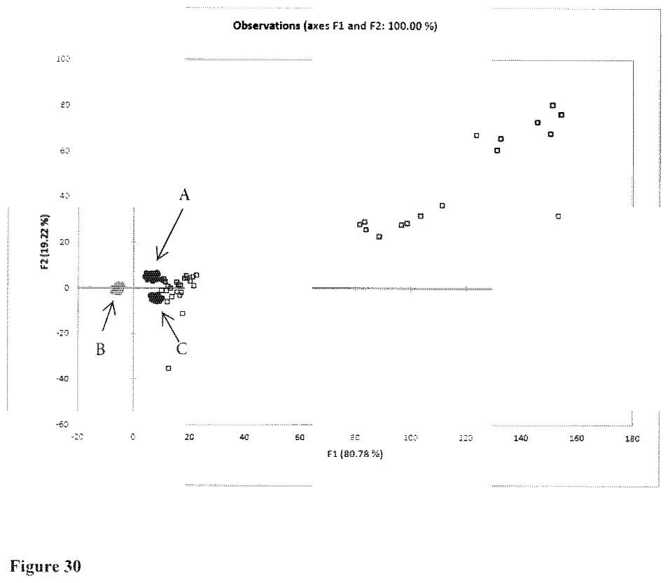

Assignment of swine offal to the correct region of origin using a muscle-specific data base prior to normalization shows that 35% of samples were correctly classified to the Western Australian Region of Origin (Table 11). All misclassifications were assigned to the North-Eastern Australia region of origin (FIG. 30). It would be recognised that the success of the classification depended on the closeness of the tissue type to muscle in respect to their multi-elemental profile. Of the samples correctly classified 50% were tongue samples while none were liver or kidney. These findings are not unexpected given the tongue was found to be the most closely related tissue type to muscle while liver and kidney were found to be the least equivalent.

TABLE 11
Percentage of correctly predicted growing regions of origin for swine
tongue, stomach, heart, liver and kidney using a muscle-specific database.
Raw values, indicate chemical profiles without mathematical correction
and normalised indicated chemical profiles of tissue post normalisation
to their muscle-equivalent concentration using multiplication factors.

| | | Tongue | Stomach | Heart | Liver | Kidney | Total |
|---|---|---|---|---|---|---|---|
| Region of Origin | Raw | 88 | 63 | 25 | 0 | 0 | 35 |
| | Normalised | 88 | 88 | 100 | 100 | 50 | 85 |
| Farm of Origin | Raw | 25 | 25 | 0 | 0 | 0 | 10 |
| | Normalized | 75 | 25 | 100 | 50 | 38 | 58 |

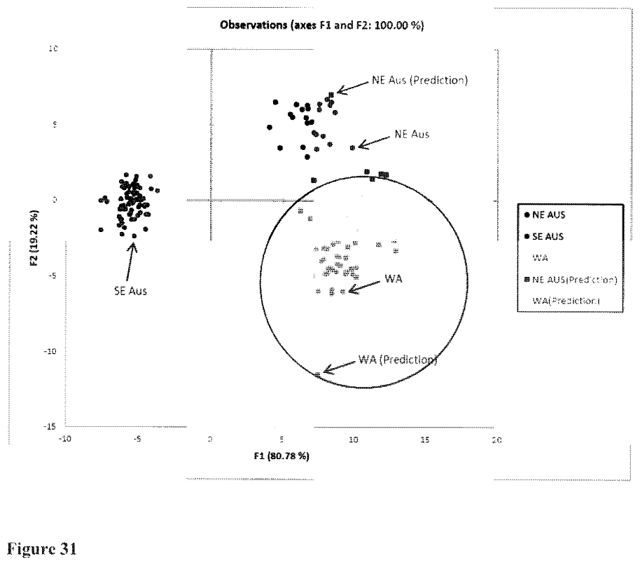

Post normalisation to their muscle-equivalent concentrations, classification of offal to the correct region of origin was considerably improved for the Western Australian samples analysed with 85% of offal correctly classifying to a Western Australian region of origin (Table 11, up from 35%). Once again all misclassified samples were assigned to the North-Eastern Australian region of origin (FIG. 31). Of those misclassified 67% were kidney samples.

As discussed previously, the normalisation factors used to correct kidney signatures are more substantial than any other tissue type. For example, a normalisation factor as low as 0.0035 for Cd in the kidney when compared to 0.32 for cadmium in the tongue is observed. Also, the relative standard deviations associated with the production of the normalisation factors are higher for kidney than all other tissue types. It would therefore be recognised that it is more difficult to associate kidney back to geographic region of origin, based on a muscle-specific data base.

Furthermore, the complex functioning of the kidney and liver should be taken into account when interpreting results as their physiology can result in an unpredictable chemical signature. The filtering and storage abilities of both the liver and kidney mean that with an increase in the concentration of ingested elements, accumulation in these organs will occur at an increased rate. However, accumulation in other organs will only increase should the ingested metal concentration rise above the capacity of the liver and kidney to process it. The complex homeostatic and filtration functions the kidney provides the body can result in a non-linear relationship between the concentration of elements in the kidney and that in the muscle, resulting in a lower ability to normalise these samples to their muscle equivalent concentrations.

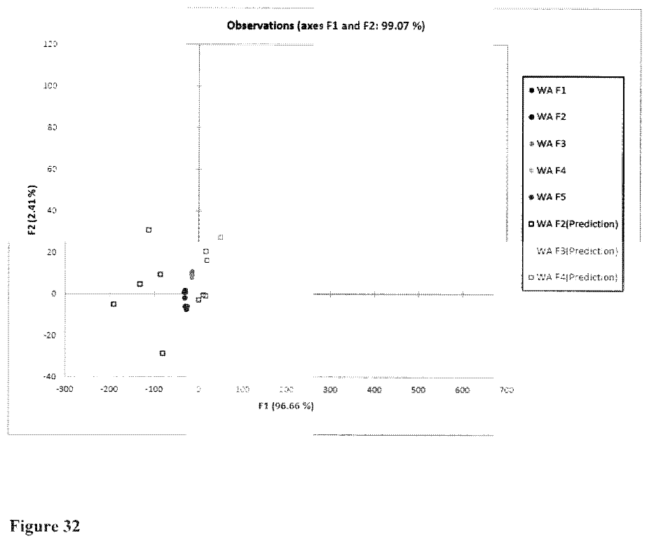

Assignment to farm of origin was also investigated for those samples that were correctly classified as originating from the Western Australian region of origin. Of the non-normalised samples correctly assigned to Western Australian region of origin, 29% of the tongue samples, 40% of the stomach samples and 0% of the heart samples were correctly classified to Western Australian Farm 3. This resulted in an overall correct classification of 25% for both tongue and stomach given prior classification to region is accounted for (Table 11). All tongue samples that were misclassified were assigned to Western Australia Farm 2 (WA F2)—see FIG. 32. For all offal data, correct assignment of samples to farm of origin was low prior to correction to a muscle-equivalent concentration.

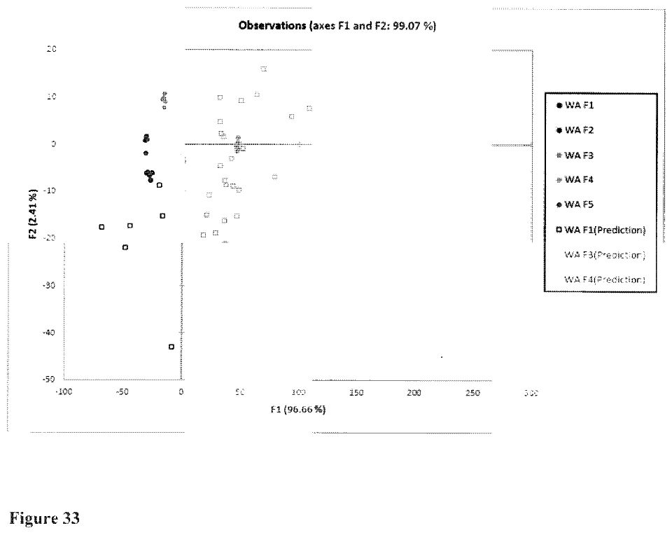

Normalisation of offal data using the developed multiplication factors resulted in an increased correct assignment of farm of origin for all tissue types except stomach (Table 11). The largest improvement was seen for heart samples which increased from 0% correct assignment to 100% correct assignment following normalisation. Correct assignment to region of origin (though not farm of origin) was increased using the normalisation factors for stomach tissue and, as such, the findings do still indicate the potential of the normalisation factors. A low level of correct assignment to farm of origin for liver and kidney was observed. This suggests that farm of origin assignment is not currently possible for these tissue types using a muscle-specific database given the substantial multiplication factors and unpredictable chemical signatures involved with these two tissue types. For all misclassified samples, assignment was either attributed to Western Australia Farm 1 (WA F1) or Western Australia Farm 4 (WA F4)—see FIG. 33.

Prediction of region and farm of origin for various swine tissues, based on a muscle-specific data base, demonstrated considerable improvement in correct classification, post normalization. For all tissue types except kidney, correct assignment to region of origin exceeded 88%, demonstrating the potential for the use of multiplication factors to enable the traceability of edible swine tissue using a muscle-specific database. For kidney, further research is required to improve classification. Based on the currently developed factors, accurate assignment to farm of origin is only possible for heart tissue. Low inter-farm variation in signatures between closely related farms, causing inability to fully separate farms, may be the underlying cause for this low assignment to farm of origin for non-heart tissue. The application of normalisation factors to the data only adds more error to the signature entered into the model thus increasing the intra-farm variation observed. In situations where this error increases the within farm variation to levels greater than the between farm variation, the chemical signatures of farms will begin to overlap minimising the distinction between farms. It would be recognised that it is for this reason that the level of classification possible for offal post normalization is lower than that possible for muscle both in the Physi-Trace™ muscle-specific database and for offal in the offal tissue-specific database.

Tracing of Unknown Offal Samples

To assess the application of the normalisation factors for the geographic assignment of an unknown offal sample to region of origin all tissue samples from three unknowns originating from Western Australia, Queensland or New South Wales were used. All tissue samples from the three unknowns were correctly assigned to their processor of origin (Table 12). This finding suggests that the normalisation factors applied to the tongue, stomach, heart, liver and kidney are appropriate for the integration of multi-elemental data of swine offal tissue into the Physi-Trace™ database. The correct classification of all tissue types from the three unknowns to processor of origin shows that a single muscle specific database can be used for the traceability of swine offal. This would facilitate a more commercially acceptable and feasible solution for the chemical traceability of swine product to a broad geographic region of origin/processor of origin as a single chemical database could be used to provide a fast and rapid means of tracing samples.

TABLE 12
Predicted processor of origin for swine muscle, tongue,
stomach, heart, liver and kidney unknown samples.

| Unknown | | | | | | |
|---|---|---|---|---|---|---|
| Processor | | | | | | |
| of Origin | Muscle | Tongue | Stomach | Heart | Liver | Kidney |
| Plant A | Plant A | Plant A | Plant A | Plant A | Plant A | Plant A |
| Plant B | Plant B | Plant B | Plant B | Plant B | Plant B | Plant B |
| Plant C | Plant C | Plant C | Plant C | Plant C | Plant C | Plant C |

For all unknowns investigated, the correct farm of origin was identified in the sorting algorithm and formed part of the linear discriminant model. The most successful classification was achieved for the samples from Farm XXX from Plant C (Table 13). For this swine, muscle, tongue, stomach and heart were all correctly classified to Farm XXX. The higher percentage of samples correctly classified from this unknown compared to the others is largely a result of this tattoo or farm accounting for a large percentage of the samples incorporated into the LDA, whereas samples from the other farms were less well represented (e.g. Farm CBC was represented by only three sample points).

TABLE 13
Farm of Origin assignment for muscle, tongue, stomach,
heart, liver and kidney from three swine. Correct
classifications are indicated by bold underlined text.

| | Plant A | Plant B | Plant C |
|---|---|---|---|
| | (AAD) | (BEB) | (CCC) |
| Muscle | AAA | BBB | CCC |
| Tongue | AAA | BAB | CCC |
| Stomach | AAB | BCB | CCC |
| Heart | AAC | BDB | CCC |
| Liver | AAB | BEB | CBC |
| Kidney | AAB | BFB | CBC |

No offal tissue samples for the Farm AAD (Plant A) unknown were correctly assigned to farm of origin (nor were muscle tissue samples). It may be that the results were negatively impacted by variation in the time of sampling or by modelling parameters, not normalisation factors. Temporal variation in sampling the unknown when compared to the sampling time of representative muscle samples for Farm AAD is a possible cause of the misclassification of tissue samples. Only 9% of samples in the Physi-Trace™ database were collected within a 2 month period of the sampling of the unknown tissues. All other representatives were collected greater than 6 months either side. This temporal variation is long enough to adversely affect the ability to correctly classify Farm AAD offal samples even with a correct normalisation to a muscle specific concentration.

Based on the farms selected by the sorting algorithm, the LDA model developed may also have contributed to the lower classification of unknown samples to farm of origin than expected. No clear separation of the different farms was achieved by LDA with a leave one out cross validation of only 55.35% determined. This was due to the large spread of sampling dates for the farms causing a high degree of intra-farm variation by incorporating chemical profiles that have changed slightly between the sampling periods. The probability of being associated with the correct farm of origin was found to be: muscle (14.9%), tongue (5.8%), stomach (0.2%), heart (30.7%), liver (12.1%) and kidney (25.2%). These findings suggest normalisation factors did reduce the offal tissue into line with the muscle tissue chemical signature, and indicates that with a more temporally appropriate database producing more clearly defined groups, an improvement in the correct assignment of the different tissue samples to farm of origin, in general, and more specifically for the Farm AAD unknown could be achieved.

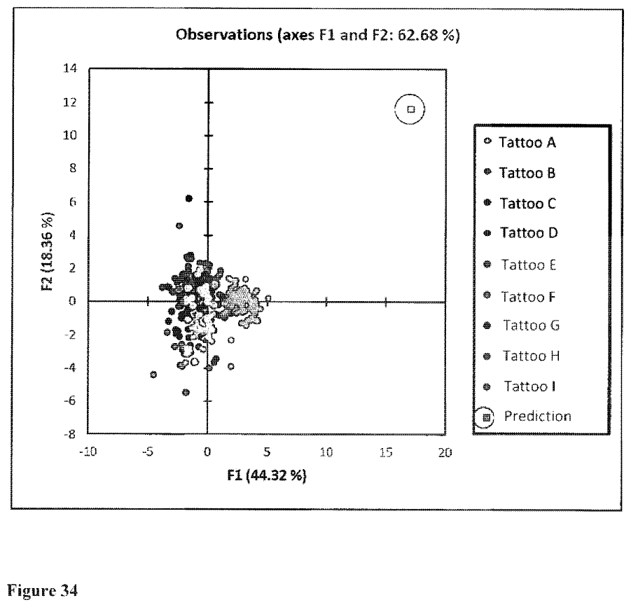

Only the liver tissue was correctly classified to farm of origin for the Farm BBB unknown. All other tissue samples were misclassified with the modelling indicating no association to the correct farm of origin. For all misclassified samples, the unknown was plotted well away from all farms identified during the sorting algorithm. This included the muscle, the association to the farms of which is demonstrated in FIG. 34. If the muscle were to associate with the farms, the misclassification of the offal tissues could be attributed to the normalisation factors used to convert the concentrations to muscle-specific values for the sample. However, as the muscle is also dissimilar to all farms used for LDA, despite the sorting algorithm selecting them as chemically similar, it is possible that an inaccurate batch correction may be limiting the ability to assign BBB samples correctly to the farm of origin.

All unknowns from the BBB pig were not run in a single batch, as they formed part of the wider offal database collected as part of this research. As such, each tissue type from the unknown swine was digested, analysed and interpreted with a different analytical group of samples. To ensure correct cross validation the whole batch was then corrected in to the pooled database. For the Plant B data, including those containing the Farm BBB unknown samples, analysis was conducted in much smaller batches than other analytical runs, with a single analysis for each tissue type. These were then corrected first to each other to produce a single Plant B database, and then into the pooled data. Furthermore, they were also the first samples to be run and could not be re-run because the samples were used to undertake offal homogeneity studies and were exhausted.

Consequently, the greater manipulation of data required for this set of samples may have introduced greater data imprecision in the data for the Plant B Farm BBB unknown than that which was associated with the other unknown samples. These factors may therefore have limited the ability to correctly assign the samples to farm of origin even when the normalisation factors were appropriate.

### Example 4

Traceability of Processed Food Samples

The aim of the study presented in Example 4 was to determine whether Australian pork products can be discriminated from non-Australian pork products.

Sample Collection

Collection of processed meat samples was carried out as follows:

(1) For each sample, collect about 100 g in a zip lock bag.

(2) Please ensure that the bag is closed tightly and as much air is squeezed out as possible

(3) On the zip lock bag, affix a label to the bag and write the date of collection, producer & origin on the blank half of the label.

(4) In a spreadsheet record the following:

(a) Date of sampling

(b) Physi-Trace™ number

(c) Producer

(d) Origin of the meat

(e) Type of ham or bacon—i.e., double smoked ham, honey ham etc.

(5) Put the sample in an portable cooler (an esky) with an ice brick.

(6) Freeze samples if they need to be stored.

Sample Preparation for Analysis

Labelled sample bags, containing samples of processed meat (Canadian bacon, Australian bacon and Australian ham), were received from a quarantine approved premises for importation of raw pork. A sub-sample (approximately 2 g wet mass) of processed meat was placed into labelled, pre-weighed 50 mL centrifuge tubes and digested. An acid/peroxide solution (5 mL HNO₃ and 2 mL H₂O₂) was added to each tube and the tubes placed onto a water bath at 50° C. overnight. The water bath temperature was then increased to 90° C. and the samples evaporated to approximately 1 mL volume. The samples were then made up to 30 mL using high purity 18 MegΩ MilliQ water.

Reagent blanks were included in each water bath rack (minimum of 3). CRM DOLT-4 and TORT-2 and NCS DC73347, and an in-house freeze dried pork standard were also included in each digestion rack to ensure accuracy and facilitate data normalisation. Approximately 0.2-0.5 g of each CRM and freeze dried standard were accurately weighed into 50 mL centrifuge tubes and digested in the same manner as the processed food samples. Analysis was carried out as discussed in Example

Discrimination Between Australian and Non-Australian Processed Meat Products

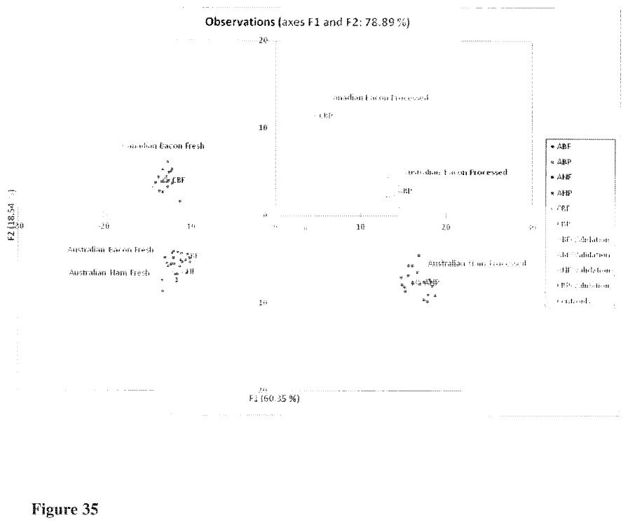

Data relating to processed meat products were classified according to their country of origin (Australian and Canadian) and their stage of processing (fresh, bacon and ham). LDA was undertaken on this data, generating a discriminant model based upon 28 analytes which correctly classified 100% of the samples in the data set (100% correct cross validation). The discriminant plot associated with this model is detailed in FIG. 35, illustrating separation between all data groups (both fresh and processed as well as Canadian and Australian) with a close association between Australian fresh (unprocessed) bacon and Australian fresh (unprocessed) ham. These results are consistent with those of previous studies which indicate that the elemental compositions of meat from various parts of the animal are similar and that discrimination can easily be achieved between Australian and non-Australian pork products.

### Example 5

24-Hour Traceback Exercise

In the event of a food safety incident, a rapid turnaround time would be required for a traceback to ensure minimal overall disruption to the Australian pork industry. An investigation was initiated to establish if the 24-hour time frame required by legislation could be achieved.

Two samples of known origin were selected from those provided for the National Livestock Traceability Performance Standards Project. The identity of those samples was concealed from the scientists undertaking the analysis and only disclosed once results had been established. A timeline of the traceback process is listed in Table 14, detailing the procedures undertaken and the point at which classification was achieved. Timing commenced following receipt of the sample at 17:00 h on Day 1 and concluded upon delivery of the results at approximately 11:00 h on Day 2.

TABLE 14
Timeline for 24-hour traceback exercise.

| Time | |
|---|---|
| (hh:min) | Description |
| −00:45 | Preparation of laboratory equipment and QA/QC samples |
| 00:00 | Sample received |
| 00:15 | Sample digestion commenced |
| 10:00 | Lids removed from sample vessels |
| 12:30 | First samples conclude digestion, instrumentation started |
| 14:00 | Sample digestion completed, analysis commences on |
| | ICP-MS/AES |
| 15:30 | ICP-AES analysis completed |
| 15:45 | ICP-AES data processing completed, ICP-MS analysis |
| | completed |
| 16:30 | ICP-MS data processing completed |
| 17:00 | Data combined and normalised to database |
| 17:30 | Unknown samples classified to processors |
| 18:00 | Potential tattoo codes of origin determined and confirmed |
| | by second scientist |

Classification of the two unknown samples to their processor of origin was achieved using a discriminant model based upon 46 analytes that correctly classified 95.6% and 96.6% of the estimation and validation samples respectively. Unknown 1 was predicted to have originated from Plant B while unknown 2 was predicted to have originated from Plant A. These classifications were subsequently confirmed. Further interpretation of the results showed that an unknown could be traced to a confirmed tattoo code with classification statistics (19 analytes, 79.1% and 70.6% correct estimation and validation samples). It would be appreciated that the statistics would be higher through using a more current reference database.

The results show that under the conditions of the exercise it was it was possible to undertake the scientific and analytical process of a traceback investigation in 18 hours from an operational perspective. This is within the 24-hour requirement of the legislation.

The person skilled in the art would recognise that preparative laboratory work on the scale of 45 minutes as used in this example exercise would normally be available to a scientist and undertaken prior to receipt of samples in response to a food safety incident. It would also be recognised that the exercise conditions are within the bound of a real food safety traceback event where success is dependent on both the currency of the database and also the resourcing of the investigation.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The claims defining the invention are as follows:

1. A method of reporting the identity of an unknown pig sample, the method comprising:
a) registering a plurality of samples referenced to an individual pig animal or a group of pig animals in a database;
b) recording data representing the plurality of reference samples against the register;
c) recording data representing the unknown pig sample in the database;
d) comparing the data representing the unknown pig sample with the data representing the plurality of reference samples to thereby assess the identity of the unknown pig sample through the register; and
e) generating a report providing an assessment of the identity of the unknown pig sample.

2. The method according to claim 1, wherein the identity is selected from the country of origin, the region of origin, the state of origin, the producer, the processor, or the property of origin.

3. The method according to claim 2, wherein the property of origin is linked to a tattoo, property identification code (PIC) and/or Australian Pork Limited (APL) PigPass Registration Number.

4. The method according to claim 1, wherein the identity is the individual pig animal.

5. The method according to any one of claims 1 to 4, wherein the plurality of samples referenced to an individual pig animal or a group of pig animals is a plurality of reference samples taken from pig tissue.

6. The method according to claim 5, wherein the pig tissue is muscle.

7. The method according to claim 5, wherein the pig tissue is offal.

8. The method according to any one of claims 1 to 4, wherein the plurality of samples referenced to an individual pig animal or a group of pig animals is taken from a pork product.

9. The method according to claim 8, wherein the pork product is a processed pork product.

10. The method according to claim 9, wherein the processed pork product is selected from whole muscle bacon or ham.

11. The method according to any one of claims 1 to 10, wherein the method further comprises a sampling protocol for determining the number of samples to be taken for analysis.

12. The method according to claim 11, wherein the sampling protocol is based on the number of pigs killed in a given week at an abattoir.

13. The method according to claim 12, wherein the sampling protocol is based on the number of pigs killed in a given week at an abattoir and the number of unique tattoos that appear in the given week.

14. The method according to any one of claims 11 to 13, wherein a sample of about 5 g to 10 g is collected.

15. The method according to any one of claims 11 to 14, wherein a small percentage of all samples taken is randomly selected and submitted for analysis.

16. The method according to claim 15, wherein about 0.1% to about 5% of all samples taken are randomly selected and submitted for analysis.

17. The method according to claim 10, wherein the method further comprises a sampling protocol based on the number of ham or bacon samples sourced from Australia per month.

18. The method according to claim 17, wherein a sample of about 20 g is collected.

19. The method according to claim 17 or claim 18, wherein about 10% of ham or bacon samples sourced from Australia are submitted for analysis.

20. The method according to claim 10, wherein the method further comprises a sampling protocol where no less than 5 ham and 5 bacon samples are taken from product manufactured from pork sourced from within a region within a country other than Australia every month.

21. The method according to claim 20, wherein a sample of about 20 g is collected.

22. The method according to claim 20 or claim 21, wherein about 10% of all samples taken are submitted for analysis.

23. The method according to any one of claim 15, 16, 19 or 22, wherein sub-samples of samples submitted for analysis are analysed by a solution-based method.

24. The method according to claim 23, wherein the solution-based method is spectrometric and/or spectroscopic.

25. The method according to claim 23 or claim 24, wherein the sub-samples are chemically digested to allow subsequent analysis.

26. The method according to claim 25, wherein the sub-samples are chemically digested with a mixture of nitric acid and hydrogen peroxide.

27. The method according to any one of claims 23 to 26, wherein the sub-samples are analysed for metals and/or non-metal elements selected from the group consisting of:
sodium, magnesium, silicon, phosphorus, sulphur, potassium, calcium, manganese, iron, copper, zinc, aluminium, scandium, lithium, beryllium, boron, titanium, vanadium, chromium, cobalt, nickel, gallium, germanium, arsenic, selenium, rubidium, strontium, yttrium, zirconium, niobium, molybdenum, ruthenium, rhodium, palladium, silver, cadmium, indium, tin, antimony, tellurium, caesium, barium, lanthanum, cerium, praseodymium, neodymium, samarium, europium, gadolinium, terbium, dysprosium, holmium, erbium, thulium, ytterbium, lutetium, hafnium, tantalum, tungsten, gold, rhenium, iridium, platinum, mercury, thallium, lead, bismuth, thorium, and uranium.

28. The method according to claim 27, wherein the metals and/or non-metal elements are analysed by Inductively Coupled Plasma Atomic Emission Spectrophotometry (ICP-AES) with the exception of lanthanum, cerium, praseodymium, neodymium, samarium, europium, gadolinium, terbium, dysprosium, holmium, erbium, thulium, ytterbium and lutetium.

29. The method according to claim 28, wherein sodium, magnesium, silicon, phosphorus, sulphur, potassium, calcium, manganese, iron, copper, zinc, aluminium and scandium are analysed by ICP-AES.

30. The method according to claim 27, wherein lithium (Li), beryllium (Be), boron (B), aluminium (Al), scandium (Sc), titanium (Ti), vanadium (V), chromium (Cr), cobalt (Co), nickel (Ni), copper (Cu), zinc (Zn), gallium (Ga), germanium (Ge), arsenic (As), selenium (Se), rubidium (Rb), strontium (Sr), yttrium (Y), zirconium (Zr), niobium (Nb), molybdenum (Mo), ruthenium (Ru), palladium (Pd), silver (Ag), cadmium (Cd), indium (In), tin (Sn), antimony (Sb), tellurium (Te), caesium (Cs), barium (Ba), lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), tantalum (Ta), tungsten (W), mercury (Hg), thallium (Tl), lead (Pb), bismuth (Bi), thorium (Th) and uranium (U) are analysed by Inductively Coupled Plasma Mass Spectrometry (ICP-MS).

31. The method according to claim 27, wherein vanadium (⁵¹V), chromium (⁵³Cr), and arsenic (⁷⁵As) are analysed by Inductively Coupled Plasma Collision Cell Mass Spectrometry (ICP-CC-MS).

32. The method according to any one of claims 27 to 31, wherein analysis is monitored to correct for instrumental drift.

33. The method according to any one of the claims 25 to 32, wherein chemical digestion and analysis of the sub-samples is run in parallel with at least one standard sample of known composition and known weight for assessing the quality of the analytical data.

34. The method according to any one of claims 27 to 33, wherein for each analytical batch run fifteen percent of the sub-samples are analysed in duplicate to determine reproducibility of the analytical data.

35. The method according to claim 34, wherein a minimum of three cross-over samples from a previous batch run are incorporated into the batch run to determine batch variation between analytical runs.

36. The method according to claim 35, wherein the analytical data is processed.

37. The method according to claim 36, wherein the processing comprises filtering analytical data to provide a completed concentration dataset.

38. The method according to claim 37, wherein the processing comprises the step of filtering ICP-MS analytical data to provide a completed ICP-MS concentration dataset.

39. The method according to claim 37, wherein the processing comprises the step of filtering ICP-CC-MS analytical data to provide a completed ICP-CC-MS concentration dataset.

40. The method according to claim 37, wherein the processing comprises the step of filtering ICP-AES analytical data to provide a completed ICP-AES concentration dataset.

41. The method according to any one of claims 37 to 40, wherein filtering analytical data comprises the steps of:
recognition of missing or compromised data;
adjustment of analytical data depending on counts per second (cps) recorded;
assessment of calibration standards;
correcting for interference from isobaric overlap and polyatomic interference;
identifying and correcting analytical trains;
drift correction;
blank correction;
concentration calculation from cps data;
calculation of detection limit and limit of determination;
removal of ancillary laboratory solutions and internal standards;
identification and selection of preferred isotopes when the analytical technique is ICP-MS or ICP-CC-MS;
identification and selection of preferred wavelength when the analytical technique is ICP-AES;
assessing the measured values for standard samples;
comparison of duplicate samples;
comparison of replicate samples;
comparison of crossover samples;
rechecking of standards following crossover correction;
merging of ICP-AES, ICP-MS and/or ICP-CC-MS datasets; and
cross comparison of ICP-AES, ICP-MS and/or ICP-CC-MS data to provide a final completed concentration dataset.

42. The method according to claim 41, wherein all analytical data which is recorded with a value of less than one cps is replaced with a value of 1.00 when the analytical technique is ICP-AES.

43. The method according to claim 42, wherein the ICP-MS analytical data is corrected for errors associated with isobaric overlap and/or polyatomic interferences.

44. The method according to any one of claims 41 to 43, wherein the final completed concentration dataset is processed to provide data representing the plurality of reference samples in the form of multi-elemental concentration profile representing the plurality of reference samples.

45. The method according to claim 44, wherein the multi-elemental concentration profile is presented as parts per billion (ppb) or parts per million (ppm) of each element based on the dry weight of the sub-samples.

46. The method according to claim 45, wherein a statistical tool is used to process the multi-elemental concentration profiles representing the plurality of reference samples.

47. The method according to claim 46, wherein the statistical tool is a multivariate statistical tool selected from the group consisting of: linear discriminant analysis (LDA), principle component analysis (PCA), Wards method of hierarchical clustering, multinominal models (MN), support vector machines (SVM), mixture discriminate analysis (MDA), classification tree (CT) and neural networks (NN).

48. The method according to claim 47, wherein the multivariate statistical tool is LDA.

49. The method according to claim 48, wherein the LDA is conducted using a forward step-wise model.

50. The method according to claim 49, wherein the model has a tolerance level of 0.00001 and a significance level of 5%.

51. The method according to claim 49 or claim 50, wherein the accuracy of the LDA model is tested using a cross validation process.

52. The method according to claim 51, wherein the cross validation process comprises the comparison of raw analytical data.

53. The method according to claim 52, wherein the comparison of raw analytical data comprises the comparison of elemental associations.

54. The method according to claim 51, wherein the cross validation process is a leave-one-out cross validation.

55. The method according to any one of claims 36 to 54, wherein processing the analytical data further comprises standardising of multi-element concentration profiles for offal tissue data to multi-elemental concentration profiles for muscle tissue data to allow use of a single database for all raw pig tissues.

56. The method according to claim 55, wherein the standardising comprises the calculation of multiplication factors that enable the normalisation of the chemical concentration in offal tissue back to muscle-equivalent concentrations.

57. The method according to any one of claims 36 to 56, wherein processing the analytical data further comprises standardising of multi-elemental concentration profiles for processed foodstuff samples to multi-elemental concentration profiles for muscle tissue samples to allow use of a single database for all pig samples.

58. The method according to any one of claims 1 to 57, wherein the unknown pig sample is about 10 g.

59. The method according to claim 58, wherein sub-samples of the unknown pig sample are analysed as defined in any one of claims 25 to 57 to provide data representing the unknown pig sample.

60. The method according to claim 59, wherein LDA is used in the step of comparing the data representing the unknown pig sample with the data representing the plurality of reference samples to thereby identify the unknown pig sample.

61. The method according to claim 60, wherein the step of comparing comprises integrating the data representing the unknown pig sample with the data representing the plurality of reference samples and conducting LDA using a forward step-wise model to thereby identify the unknown pig sample.

62. The method according to any one of claims 58 to 61, wherein the report may be generated within about 24 hours of commencing digestion of a sub-sample of the unknown pig sample.
